(19) United States
(12) Patent Application Publication
(10) Pub. No.: US 2008/0193440 A1
(43) Pub. Date: Aug. 14, 2008

(54) Method For Multiple Sclerosis Treatment and Prophylaxis By Treatment of Leptospira Infection

(75) Inventor: Per Moestrup Jensen, Kobenhavn Nv (DK)

Correspondence Address: DARBY & DARBY P.C., P.O. BOX 770, Church Street Station, New York, NY, 10008-0770, US

(73) Assignee: Kobenhavns Universitet, Frederiksberg C (DK)

(21) Appl. No.: 11/571,478

(22) PCT Filed: Jun. 29, 2005
(86) PCT No.: PCT/DK05/00444

§ 371 Date: Apr. 17, 2008

(30) Foreign Application Priority Data
Jul. 1, 2004 (DK) PA 2004 01042

(51) Int. Cl. A61K 39/02; A61K 39/40; A61K 39/385; A61K 39/44; A61K 38/20; A61K 38/21; A61K 31/165; C12Q 1/00; G01N 33/53

(52) U.S. Cl. 424/131.1; 424/234.1; 424/178.1; 424/193.1; 424/85.2; 424/85.4; 514/619; 435/4; 435/7.1

(57) ABSTRACT

The present invention provides novel therapies and novel prophylactic methods for multiple sclerosis. Also provided are novel diagnostic methods. The invention resides in the finding that multiple sclerosis most likely is caused by chronic infection with a microorganism that cross-reacts immunologically with the spirochete Leptospira interrogans, which normally infects the brown rat.

### FIELD OF THE INVENTION

The present invention pertains to the field of treatment, prophylaxis, diagnosis and monitoring of multiple sclerosis (MS). In particular, the present invention suggests treatment and prophylaxis of MS by targeting a causative agent related to the brown rat, Rattus norvegicus. In this context the present invention provides treatment of humans by targeting the spirochete Leptospira, notably L. interrogans, which endemically infects rats.

### BACKGROUND OF THE INVENTION

### Current Knowledge on Multiple Sclerosis

The following background description of MS is to a great extend a direct quotation from the Multiple Sclerosis International Federation homepage www.msif.org. When including other sources of information a reference is given.

MS is an exceptionally difficult disease to research for a number of reasons:
The cause is unknown, though it is generally believed to be a combination of genetic, immunological and environmental factors. However, because it often takes many years for someone to be diagnosed, and because there are so many variables, it has so far been impossible to determine a specific cause or trigger for MS.
The effects are within two of the most inaccessible parts of the body, the brain and the spinal cord. It is only since the advent of Magnetic Resonance Imaging (MRI), in the early 1980s, that scientists have actually been able to view the lesions within the brain and spinal cord.
There is no single pattern to the disease, indeed there are four types of MS: relapsing-remitting, progressive relapsing, primary progressive and secondary progressive.

The course of the disease is unpredictable. The number and position of lesions on a patient's central nervous system does not necessarily correlate with their relapse occurrence or level of disability. There are no definitive tests for the disease.

### Pathogenesis

Multiple sclerosis is one of the most common diseases of the central nervous system (brain and spinal cord). MS is an inflammatory demyelinating condition. Myelin is a fatty material that insulates nerves, acting much like the covering of an electric wire and allowing the nerve to transmit its impulses rapidly.

In multiple sclerosis, the loss of myelin (demyelination) is accompanied by a disruption in the ability of the nerves to conduct electrical impulses to and from the brain and this produces the various symptoms of MS. The sites where myelin is lost (plaques or lesions) appear as hardened (scar) areas: in multiple sclerosis these scars appear at different times and in different areas of the brain and spinal cord. The term multiple sclerosis means, literally, many scars.

The damage to myelin in MS has been considered to be due to an abnormal response of the body's immune system, which normally defends the body against invading organisms (bacteria and viruses). Many of the characteristics of MS suggest an ‘auto-immune’ disease whereby the body attacks its own cells and tissues, which in the case of MS is myelin. Researchers do not know what triggers the immune system to attack myelin, but it is thought to be a combination of several factors.

One theory that has been brought forward is that a virus, possibly lying dormant in the body, may play a major role in the development of the disease and may disturb the immune system or indirectly instigate the auto-immune process. A great deal of research has taken place in trying to identify an MS-causing virus. As a consequence of this research, it is today considered probable that there is no one single MS virus, but that a common virus, such as measles or herpes, may act as a trigger for MS. This trigger activates white blood cells (lymphocytes) in the blood stream, which enter the brain by making vulnerable the brain's defence mechanisms (i.e. the blood/brain barrier). Once inside the brain these cells activate other elements of the immune system in such a way that they attack and destroy myelin.

The course of MS is unpredictable. Some people are minimally affected by the disease while others have rapid progress to total disability, with most people fitting between these two extremes. Although every individual will experience a different combination of MS symptoms there are a number of distinct patterns relating to the course of the disease.

### Types of MS

Relapsing-Remitting MS: In this form of MS there are unpredictable relapses (exacerbations, attacks) during which new symptoms appear or existing symptoms become more severe. This can last for varying periods (days or months) and there is partial or total remission (recovery). The disease may be inactive for months or years.

Benign MS: After one or two attacks with complete recovery, this form of MS does not worsen with time and there is no permanent disability. Benign MS can only be identified when there is minimal disability 10-15 years after onset and initially would have been categorised as relapsing-remitting MS. Benign MS tends to be associated with less severe symptoms at onset (e.g. sensory).

Secondary Progressive MS: For some individuals who initially have relapsing-remitting MS, there is the development of progressive disability later in the course of the disease often with superimposed relapses.

Primary Progressive MS: This form of MS is characterised by a lack of distinct attacks, but with slow onset and steadily worsening symptoms. There is an accumulation of deficits and disability which may level off at some point or continue over months and years.

### Diagnostic Tests

Current diagnostic tests include:

a) Medical History: A detailed medical history which will include past records of signs and symptoms as well as the current status of the subject.

b) Neurological Examination: A test for abnormalities in the nerve pathways that take messages from the brain to the other parts of the body. This includes changes in eye movements, limb co-ordination, weakness, balance, sensation, speech, and reflexes.

c) Testing of Visual, Auditory and Somatosensory Evoked Potentials: Measures of the speed at which messages from the brain pass along the nerves.

d) Magnetic Resonance Imaging (MRI): The MRI clearly shows the size, quantity and distribution of lesions and together with supporting evidence from medical history and neurological examination, is very significant indicator toward confirming the diagnosis of MS. It is abnormal in over 95% with a definite clinical diagnosis. The MRI is a very useful tool in clinical trials in assessing the value of new therapies, due to its ability to demonstrate changes in the disease's activity.

e) Lumbar Puncture: The proteins in the spinal fluid of the majority people (90%) with established MS form a particular pattern when an electrical current is passed through them, and so this procedure can potentially confirm an MS diagnosis.

### Recognised Treatment for MS

As yet there is no cure for MS but there are facets of the disease which have recognised treatments and which can be very effective

Exacerbations: The standard treatment for significant acute exacerbations is the use of steroids, which exert powerful anti-inflammatory effects. Steroids reduce inflammation at the site of new demyelination, allowing return to normal function to occur more rapidly and reducing the duration of the exacerbation. The current favoured steroid regimen is methylprednisolone given intravenously in high doses for 3-5 days with, perhaps, subsequent tapering lower oral doses of prednisone for 1-2 weeks. The use of steroids are not thought to have any effect on the long-term course of the disease.

### Altering the Course of the Disease

A number of new drugs have recently been approved for use in MS which have some effect on the frequency and severity of exacerbations and the number of lesions as seen on MRI, though the effect on progression of disability remains unclear.

General Disease Modifying Therapies
Glatiramer acetate (Copaxone®)
Interferon β-1a (Avonex®)
Interferon β-1a (Rebif®)
Interferon β-1b (Betaseron® or Betaferon®)
Mitoxantrone (Novantrone®)

Acute Exacerbations
Dexamethasone (Decadron®)
Methylprednisolone (Depo-Medrol®)
Prednisone (Deltasone®)

### Symptom Specific Treatment

For many of the symptoms that occur in MS, effective treatments are available.

Spasticity
Baclofen (Lioresal®)
Clonazepam (Klonopin® or Rivotril®)
Dantrolene (Dantrium®)
Diazepam (Valium®)
Gabapentin (Neurontin®)
Tizanidine (Zanaflex®)

Tremor
Clonazepam (Klonopin® or Rivotril®)
Isoniazid (Laniazid®)

Fatigue
Amantadine
Fluoxetine (Prozac®)
Modafinil (Provigil®)
Pemoline (Cylert®)

Bladder Dysfunction
Ciprofloxacin (Cipro®)
Desmopressin (DDAVP Nasal Spray®)
Imipramine (Tofranil®)
Methenamine (Hiprex, Mandelamine®)
Nitrofurantoin (Macrodantin®)
Oxybutynin (Ditropan®)
Oxybutynin: extended release formula (Ditropan XL®)
Phenazopyridine (Pyridium®)
Propantheline bromide (Pro-Banthine®)
Sulfamethoxazole (Bactrim® or Septra®)
Tolterodine (Detrol®)

Bowel Dysfunction
Bisacodyl (Dulcolax®)
Docusate (Colace®)
Docusate mini enema (Therevac Plus®)
Glycerin (Sani-Supp Supository®)
Magnesium hydroxide (Phillips' Milk of Magnesia®)
Mineral oil
Psyllium hydrophilic mucilloid1 (Metamucil®)
Sodium phosphate (Fleet Enema®)

Sexual Dysfunction
Alprostadil (Prostin VR®)
Alprostadil (MUSE®)
Papaverine
Sildenafil (Viagra®)

Pain
Amitriptyline (Elavil®)
Carbamazepine (Tegretol®)
Clonazepam (Klonopin® or Rivotril®)
Gabapentin (Neurontin®)
Imipramine (Tofranil®)
Nortriptyline (Pamelor® or Aventyl®)
Phenyloin (Dilantin®)

Cognitive, Psychiatric and Psychological Dysfunction
Bupropion (Wellbutrin®)
Fluoxetine (Prozac®)
Paroxetine (Paxil®)
Sertraline (Zoloft®)
Venlafaxine (Effexor®)

Vertigo & Dizziness
Meclizine (Antivert® or Bonamine®)

Temperature Sensitivity & Paroxysmal itching
Hydroxyzine (Atarax®)

Nausea; Vomiting
Meclizine (Antivert® or Bonamine®)

### Rehabilitation and Management

While it may not be possible to improve all lost function, all people with MS should try to optimise their physical, mental and social condition. After an exacerbation there may be the need for restorative rehabilitation. During remission periods people with MS should participate in a maintenance therapy programme to achieve and sustain their optimum physical condition. This may involve physiotherapy, stretching, coordination exercises, speech and swallowing instruction. It may also include medication, good nutrition and counseling. There may be the need for lifestyle changes (both social and occupational).

### Alternative Therapies

Given that there is no cure for MS, there is no shortage of alternative approaches to the management of the disease. Alternative therapies generally refer to non-establishment, non-traditional and often non-medical treatments. The following are a small selection of commonly recommended alternative therapies:

Supplements and Vitamins: Adequate intake of vitamins is advised in all patients with MS, but there appears no scientific proof that supplementary doses of vitamins, or megavitamin therapy, alone or in combination, favourably affect the course of the disease.

Fatty Acids: Several clinical trials have shown that dietary supplementation with polyunsaturated fatty acids (e.g. evening primrose oil) and fatty acids of fish oil appears to exert a modest effect in slowing progression and reducing the severity and duration of MS exacerbations without affecting their frequency.

Diets: There is no credible evidence that MS is due to poor diet or dietary deficiencies. A balanced diet incorporating low-fat and high-fibre is recommended for most people and should be part of the general management of the disease for people with MS. Within this framework, diets such as Swank's low-fat diet and the Kousmine diet are acceptable particularly as the possibility of a partial or incomplete effect has not been excluded. Other diets such as Allergen-free diet; Gluten-free diet; Raw food, Evers diet; McDougal diet; Protein- and Fructose restricted diet; Cambridge and other liquid diets; Sucrose and Tobacco-free diets have not been substantiated as having any effect on the course of MS although their proponents cite much personal and individual anecdotal evidence. Many of these diets (and consultations) are expensive. They can alter the normal nutritional balance and may prove unsafe and hazardous to health without medical and professional supervision.

Replacement of Dental Mercury Restorations (fillings): The removal of amalgam restorations (which are composed of silver and mercury) from teeth is based on the unsubstantiated claim that MS results from mercury poisoning and that leakage from amalgam restorations damages the immune system. There is no evidence to suggest that removal of amalgam is of any value in MS.

Acupuncture: There is no evidence to suggest that acupuncture has an effect on the disease process or symptom management. However, acupuncture may serve a purpose for the relief of pain and muscle spasm.

Yoga & Meditation: Exercise and relaxation can be a valuable and enjoyable therapy for people with MS. Yoga and meditation can improve the quality of life for people with MS, producing better social and physical functioning. There are many organisations which run courses specially for people with physical disabilities. Contact your local MS Society for appropriate recommendations and referrals.

Hyperbaric Oxygen (HBO): The breathing of oxygen under increased pressure in a specially constructed chamber hoping to arrest the course of MS and improve symptoms became popular in the 1980's. Separate trials carried out in USA, UK, Canada and the Netherlands were unanimous that HBO has no effect on any objective Special Report

Experimental Lab Studies with Promising Effect.

In the ongoing research on the treatment of MS additional documentation of effect on MS has been forwarded for several treatment regimes. These are not at this time adopted in standard recommended or alternative treatment strategies for MS by the Multiple Sclerosis International Federation.

Evidence has emerged that statins, which are inhibitors of 3-hydroxy-3-methylglutaryl coenzyme A reductase, have immunomodulatory effects. Recent reports showed that statins prevent and reverse chronic and relapsing experimental autoimmune encephalomyelitis, an animal model of MS. Furthermore, in vitro experiments with human immune cells have shown an immunomodulatory profile of statins comparable to that of interferon β (Neuhaus O, Stuve O, Zamvil S S, Hartung H P. Are statins a treatment option for multiple sclerosis? Lancet Neurol. 2004 June; 3 (6):369-71).

Morphometric examination of neurofilament-labeled axons in the spinal cord of CR-EAE animals showed that both flecainide treatment regimens resulted in significantly higher numbers of axons surviving the disease (83 and 98% of normal) compared with controls (620% of normal). These findings indicate that flecainide and similar agents may provide a novel therapy aimed at axonal protection in MS and other neuroinflammatory disorders (Bechtold D A, Kapoor R, Smith K J. Axonal protection using flecainide in experimental autoimmune encephalomyelitis. Ann Neurol. 2004 May; 55 (5):607-16).

Estrogen treatment has been found to be protective in experimental autoimmune encephalomyelitis (EAE) and possibly multiple sclerosis (MS) (Palaszynski K M, Liu H, Loo K K, Voskuhl R R. Estriol treatment ameliorates disease in males with experimental autoimmune encephalomyelitis: implications for multiple sclerosis. J Neuroimmunol. 2004 April; 149 (1-2):84-9).

Treatment with antioxidants might theoretically prevent propagation of tissue damage in MS (Gilgun-Sherki Y, Melamed E, Offen D. The role of oxidative stress in the pathogenesis of multiple sclerosis: the need for effective antioxidant therapy. J Neurol. 2004 March; 251 (3):261-8).

A phase II open label baseline-to-treatment trial of a humanized monoclonal antibody against CD25 (daclizumab) in 10 multiple sclerosis patients with incomplete response to IFN-β therapy and high brain inflammatory and clinical disease activity demonstrated that Daclizumab was very well tolerated and led to a 78% reduction in new contrast-enhancing lesions and to a significant improvement in several clinical outcome measures (Bielekova B, Richert N, Howard T, Blevins G, Markovic-Plese S, McCartin J, Wurfel J, Ohayon J, Waldmann T A, McFarland H F, Martin R. Humanized anti-CD25 (daclizumab) inhibits disease activity in multiple sclerosis patients failing to respond to interferon β Proc Natl Acad Sci USA. 2004 May 25).

Based on patient reports, animal data, and in vitro experiments, evidence has emerged indicating a positive effect of cannabinoids as symptomatic treatment of spasticity and pain in multiple sclerosis. The recently published CAMS study was the first multicenter, randomized, placebo-controlled phase III trial to examine the efficacy of cannabinoids on symptoms related to MS. There was no treatment effect of cannabinoids on the primary outcome measure, a difference in the reduction of spasticity as assessed by the so-called Ashworth score. In contrast, significant effects on patient-reported spasticity and pain were documented (Neuhaus O, Kieseier B C, Klimke A, Gaebel W, Hohifeld R, Hartung H P. Cannabinoids in multiple sclerosis Opportunity or threat?, Nervenarzt. 2004 May 20).

To conclude, even though progress has been made in the treatment and diagnosis of MS, there is to date no art-recognized etiological agent and hence no specific treatment or diagnosis of the disease.

### OBJECT OF THE INVENTION

It is an object of the present invention to provide means and methods for combating MS as well as means and methods for accurate and early diagnosis of MS.

### SUMMARY OF THE INVENTION

The search for the mechanisms causing multiple scleroses have produced many different types of hypotheses, but none of these have been yet been successful in identifying the causal mechanism of the disease. Broadly the ideas can be divided into groups depending on whether the mechanism was thought to depend on autoimmune processes (Conlon et al., 1999) or an etiological agent. The latter group contain studies, which have searched for a human-to-human transmitted agent (Hawkes, 2002) while others have suggested food sources (Lauer, 1991) or a wild life reservoir (Fritzsche, 2002).

The present inventor has now found persuasive evidence that MS is caused by infection with an organism that endemically infects the Brown Rat, Rattus norvegicus. As will appear from examples 2 and 3, the main candidate for a causative agent is the spirochete L. interrogans, an organism which endemically infects R. norvegicus, but as will also appear from the present disclosure, it could also be a hitherto unidentified bacterial species which cross-reacts immunologically with L. interrogans and other Leptospira.

At any rate, the present invention offers a series of novel therapeutics and diagnostics in the MS area due to this finding.

The invention therefore in a first aspect relates to a method for treating or ameliorating multiple sclerosis (MS) in a human subject suffering from MS, the method comprising active immunization of said subject with an immunogenic agent that induces a therapeutically effective immune response against antigenic determinants derived from Leptospira, said immunogenic agent comprising a specific immunogen.

In a related aspect, the invention relates to a method for preventing multiple sclerosis (MS) in a human subject, the method comprising active immunization of said subject with an immunogenic agent that induces a protective immune response against Leptospira, said immunogenic agent comprising a specific immunogen.

In a first diagnostic aspect, the invention provides a method for determining whether a person is suffering from MS or has an increased risk of attracting MS, the method comprising subjecting a sample obtained from the person to a test that determines whether or not the sample contains material derived from L. interrogans, a positive determination indicating that the person has a significantly increased risk of MS compared to a subject without a positive determination.

In a second diagnostic aspect, the invention provides for a method for assessing the risk that a person is suffering from MS or will attract MS, the method comprising subjecting a sample obtained from the person to a test that determines the presence, in the sample, of antibodies generally reactive with Leptospira and antibodies specifically reactive with L. interrogans.

In a third diagnostic aspect, the present invention provides for a method for assessing the risk that a person is suffering from MS or will attract MS, the method comprising subjecting a sample obtained from the person to a test that establishes whether the person's alternative complement pathway is capable of lysing Leptospira.

The invention also provides for a method for monitoring the progress of MS in a patient, the method comprising subjecting a sample obtained from the patient to a test that quantitatively determines L. interrogans material in the sample and comparing the determination with determinations performed on later samples from the same patient.

Also provided is a method for monitoring the progress of MS in a patient, the method comprising a quantitative determination of antibodies specifically reactive with L. interrogans in a sample obtained from the patient and comparing the determination with equivalent determinations performed on later samples from the same patient.

The invention also provides for a pharmaceutical package comprising at least one container comprising an immunogenic agent capable of inducing protective immunity in humans against L. interrogans and instructions for using the immunogenic agent for treatment or prophylaxis of humans against MS.

Furthermore, the invention provides a pharmaceutical package comprising at least one container comprising an antibiotic capable of exerting a bacteriotoxic or bacteriostatic effect on L. interrogans and instructions for using the antibiotic for treatment or prophylaxis of humans against MS.

Finally, the invention also relates to a pharmaceutical kit, comprising at least one container comprising an immunogenic agent capable of inducing protective immunity in humans against L. interrogans and at least one container comprising diagnostic means that can react with L. interrogans material or react with antibodies reactive with L. interrogans.

### LEGEND TO THE FIGURES

FIG. 1 shows theoretical curves exhibited by a zoonotic disease that mirrors endemic stability.

a: schematics of an endemic stable disease with a peak in clinical disease at intermediate exposure (line) and steadily declining age of onset (dashed line).
b: Prevalence Per Unit Exposure (PPUE) plotted against exposure as expected by the normal relationship p between catch and effort in harvested populations. Fishery terminology in brackets.
c: Density dependence in incidence of a disease as result of limits in the susceptible populations and possible depletion at previously experience high incidence.

FIG. 2 shows maps of the global distribution of MS and of Rattus norvegicus.

a: The world-wide distribution of MS as provided by (www.medlib.med.utah.edu/kw/ms/mml/ms_worldmap.html).
b: The world-wide distribution of Rattus norvegicus (Gratz N. (unknown date)).

FIG. 3: Correlation graphs based on Danish data.

a: Correlation between rat exposure and MS.
b: correlation between rat exposure and leptospirosis cases.
c: Correlation between leptospirosis and MS.
All figures include 19 counties in Denmark. Results of linear regression, with forced point at 0.0 are given in the figure.

FIG. 4 shows the result of a catch effort analysis.

a: Catch effort relationship for MS and rat densities.
b: Catch effort relationship for leptospirosis and rat densities.
c: MS per leptospirosis case against assumed level of discovery of leptospirosis.

FIG. 5 shows the geographical variation of MS in Europe.

a: Variation in prevalence of MS in Europe as result of variation in latitude.
b: Variation in prevalence of MS in Europe as result of variation longitude. Based on Rosati (2001).

FIG. 6 Shows reconstruction of the epidemiology profile of MS.

a: relative variation in incidence as function of incidence.
b: Yearly incidence of MS at different levels of R. norvegicus densities (Results of linear regression, with forced point at 0,0 is given in the figure).
c: Yearly incidence in MS for males (circles) and females (crosses) when assuming 0.9:1 male:female exposure.
d: sex ratio (males yearly incidence per total yearly incidence) versus different R. norvegicus densities.

FIG. 7: Prevalence or average yearly incidence of a disease mirroring endemic stability sensitive to cross reacting avirulent types of infections.

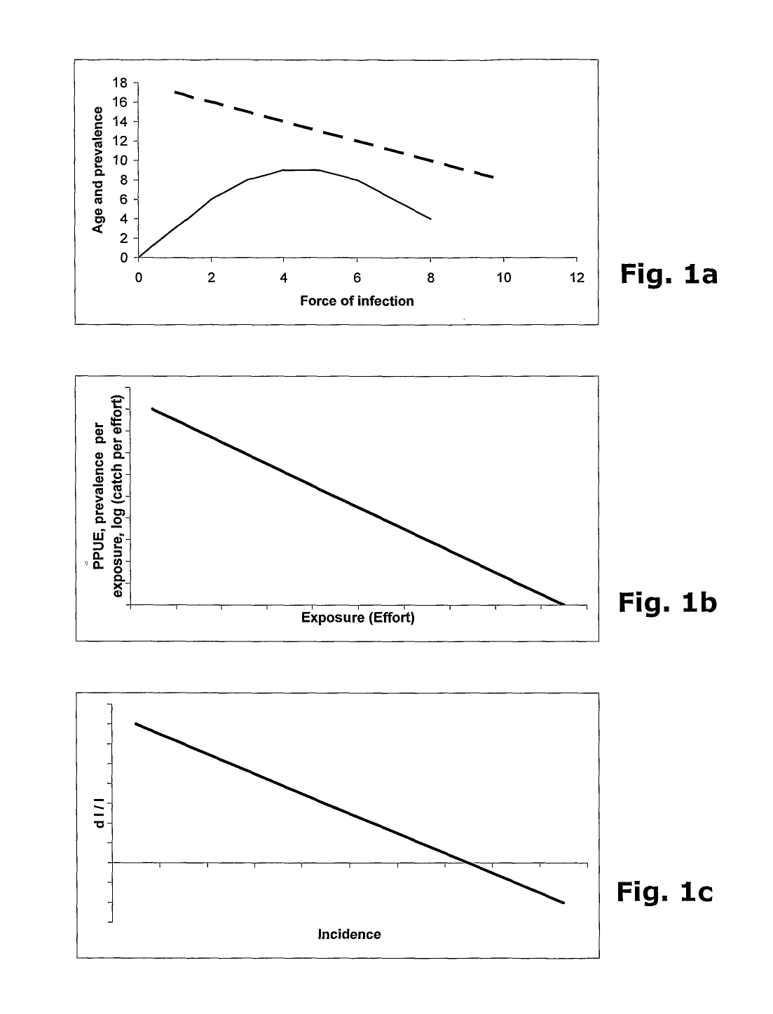

Arrows indicate increasing level of infection/inoculation rates. When avirulent cross-reacting types are at zero the relation between prevalence of disease and virulent types is as depicted in FIG. 1a.

### DETAILED DISCLOSURE OF THE INVENTION

### Definitions

In the following, a number of terms employed in the present specification and claims will be defined in order to clarify the metes and bounds of the present invention.

The term “active immunization” means induction of a specific immune response against an immunogen in an animal, meaning that the animal's own cells are primed to recognize and mount an immunological attack on the immunogen. The end-result is e.g. induction of humeral immunity, so that the animal produces antibodies that specifically reacts with the immunogen, or induction of cellular immunity so that T-cells in the animal recognize and attack cells that carry the immunogen.

A special form of active immunization is one that produces a “protective” or “therapeutic” effective immune response. By this is meant that the immunogen is so constituted that the immune response induced is capable of effectively combating a pathogenic agent. In the event the immune response is protective, the immune response is capable of arresting the development of disease caused by the pathogen before it becomes clinically relevant—this can happen at a number of levels (penetration of the pathogen may be blocked, important receptors may be blocked, proliferation of the pathogen may be blocked, the pathogen may be killed by natural killer cells or engulfed by APC upon antibody binding etc.). In the event the immune response is therapeutic, ongoing infection/disease is combated by the immune system (one example is vaccination against rabies, where the immune response develops after a person has been infected). However, both approaches rely on the quality of the immune response, meaning that the epitopes in the pathogen against which the immune response is raised must be relevant. This is normally ensured by utilising an immunogen which includes a large number of epitopes from the pathogen, as is the case when using attenuated live vaccines or killed vaccines.

An “immunogen” or “immunogenic agent” is a an agent capable of inducing a specific immune response in a certain animal or group of animals against an antigen, meaning that the term is only relevant vis-à-vis said animal or group of animals—for instance, an agent that is immunogenic in one species need not be immunogenic in another species, and an agent which is immunogenic in one individual animal need not be immunogenic in another animal of the same species. It will be understood that the agent can be an antigen as such, but the agent can also be genetic material encoding the antigen or virus or bacteria capable of expressing the antigen.

An “immunogenic composition” is a composition of matter which includes an immunogen as well as other substances that contribute to the effective in vivo use of the immunogen—such additional substances can e.g. be immunological adjuvants.

An “antigen” is a substance which in some animal is capable of inducing a specific immune response, i.e. capable of inducing antibodies or T-cells that specifically recognize the antigen.

A “hapten” is a substance which can be recognized by antibodies but which is in itself incapable of inducing antibody production.

The term “specific immunogen” is in the present context a substance or agent which, upon administration to a human, can elicit an immune response against Leptospira. The term thus embraces a number of agents that share the feature of effecting presentation of Leptospira antigenic determinants to the immune system. In this sense, the term embraces antigens derived from Leptospira, but also genetic material encoding such antigens as well as virus and non-Leptospira microorganisms that express such genetic material.

The term “immunogenically effective amount” has its usual meaning in the art of immunology, i.e. an amount of an immunogen or immunogenic composition which is capable of inducing an immune response which significantly engages molecules which share immunological features with the immunogen.

The terms “T-lymphocyte” and “T-cell” will be used interchangeably for lymphocytes of thymic origin which are responsible for various cell mediated immune responses as well as for helper activity in the humeral immune response. Likewise, the terms “B-lymphocyte” and “B-cell” will be used interchangeably for antibody-producing lymphocytes.

A “T-cell epitope” (or: “T-lymphocyte epitope”) is in the context of the present invention a peptide which is able to bind to an MHC molecule and which stimulates T-cells in humans. Preferred foreign T-cell epitopes used in the invention are “promiscuous” epitopes, i.e. epitopes which bind to a substantial fraction of a particular class of MHC molecules in humans. Only a very limited number of such promiscuous T-cell epitopes are known, and they will be discussed in detail below.

A “T helper lymphocyte epitope” (a T_{H} epitope) is a T cell epitope which binds an MHC Class II molecule and can be presented on the surface of an antigen presenting cell (APC) bound to the MHC Class II molecule. Promiscuous epitopes are functionally equivalent to immunogenic carrier proteins.

An “immunogenic carrier” is a polypeptide or protein which includes a number of T_{H} epitopes and which, when coupled to a hapten, renders the production of antibodies against the hapten possible. Because of its size and the number of different T_{H} epitopes, an immunogenic carrier is normally immunogenic is the vast majority of a human population. Examples of carriers are keyhole limpet hemocyanin (KLH), tetanus toxoid, diphtheria toxoid, and bovine serum albumin (BSA).

The term “adjuvant” has its usual meaning in the art of vaccine technology, i.e. a substance or a composition of matter which is 1) not in itself capable of mounting a specific immune response against the immunogen of the vaccine or immunogenic composition, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combination of vaccination with immunogen and adjuvant induces an immune response against the immunogen which is stronger than that induced by the immunogen alone.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. Also, the term “polyamino acid” is an equivalent to the term “polypeptide”.

The term “subsequence” means any consecutive stretch of at least 3 amino acids or, when relevant, of at least 3 nucleotides, derived directly from a naturally occurring Leptospira derived sequence.

The term “animal” is in the present context in general intended to denote an animal species (preferably mammalian), such as Homo sapiens, Canis domesticus, etc. and not just one single animal. However, the term also denotes a population of such an animal species; this is especially relevant when discussing induction of immunity, since some immunogens will not be capable of functioning effectively in all individuals of an animal species, but in spite of this, the immunogen will still exhibit its desired effect in the population where it is indeed effective.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. For instance it is possible to use certain cytokines as adjuvants that are coupled to the immunogen, and in such a case, the issue of stability may be irrelevant since the coupling provides the stability necessary.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting.

“Productive binding” means binding of a peptide to the MHC molecule (Class I or II) so as to be able to stimulate T-cells that engage a cell that present the peptide bound to the MHC molecule. For instance, a peptide bound to an MHC Class II molecule on the surface of an APC is said to be productively bound if this APC will stimulate a T_{H} cell that binds to the presented peptide-MHC Class II complex.

### Preferred Embodiments

### Methods of Prophylaxis and Methods of Treatment for MS

A direct consequence of knowing the causative agent for MS is that it becomes possible to device specific prevention and treatment for the disease. Since the evidence presented herein indicates a causative role for L. interrogans or a similar Leptospira species/strain, various measures for immunologic prophylaxis and treatment become possible, as does antibacterial pharmacotherapy. Interestingly, active immunotherapy and active immune prophylaxis utilise the same means and measures, differing only in the timing of the treatment relative to the onset of the disease.

The genome of Leptospira interrogans has been sequenced, and it is thus possible to identify a large number of protein antigens that could serve as immunogenic agents useful for prophylaxis or for active specific immunotherapy.

A listing of preferred specific immunogens include:

a) A preparation of a live Leptospira species which is non-pathogenic in humans and which preferably cross-reacts immunologically with L. interrogans—this mode of the invention can be compared to the Cadmette-Guerin vaccine used against tuberculosis, where an attenuated strain of Mycobacterium bovis is used because of it cross-reactivity with virulent mycobacteria. In this case, the most evident Leptospira strain for vaccine use would be Leptospira serovar patoc. This serovar carries virtually all the antigens of the family Leptospira.

b) A preparation of killed or inactivated L. interrogans or killed or inactivated bacteria from a Leptospira species or strain cross-reactive with L. interrogans—this mode of the invention can be compared to the most commonly used form of the diphtheria vaccine, where a inactivated preparation of a known pathogen or a close relative thereof is used for immunization. This type of vaccine is often used for vaccination against viral antigens, but also cholera vaccines utilise killed bacteria. A relevant choice for a vaccine using such immunogens would according to the present invention be “vax-SPIRAL”, a trivalent Leptospirosis Vaccine Adsorbed in Aluminum Hydroxide Gel and marketed by the Finlay Institute in Cuba.

c) An antigen fraction isolated from L. interrogans or from a cross-reactive Leptospira species or strain—this mode of the invention is very closely related to the option mentioned under b, but can include one or several purification steps where contaminants are removed by extraction, dialysis, ultracentrifugation etc, the main objective being to include a proportion of antigens from the surface of the Leptospira. Known examples from the art are e.g. vaccines against meningococcus polysaccharides.

d) A preparation of at least one antigen comprising immunodominant epitopes derived from L. interrogans or from a cross-reactive Leptospira species or strain—this mode of the invention relies on identification of immunodominant antigens from the surface of Leptospira. The most promising antigens are those known as Osp (outer surface proteins) which, in other spirochetes such as Borrelia, are known to trigger protective immunity. Since the genome of Leptospira serovar patoc is known, the cloning and recombinant expression of Leptospira Osp is a relatively easy task.

e) A preparation comprising at least one anti-idiotypic antibody reactive with the idiotype of an antibody that binds to an immunodominant epitope derived from L. interrogans or from a cross-reactive Leptospira species or strain or f) a preparation comprising at least one mimotope of an immunodominant epitope derived from L. interrogans or from a cross-reactive Leptospira species or strain—these modes of the invention require that one single or a few immunodominant epitopes are identified; in contrast to modes of the invention where a large number of Leptospira antigens are present (such as modes a-c), the immune response mounted when using a single or a few antigenic determinants in the vaccine are highly dependent on the immunodominance exerted by the epitopes. Or put in other terms, modes a-c have the inherent advantage of allowing the immune system to choose amongst a large number of putative immunogenically relevant epitopes.

g) A preparation of nucleic acids encoding and being capable of effecting in vivo expression from the subject's cells of at least one immunodominant protein antigen derived from L. interrogans or from a cross-reactive Leptospira species or strain—this mode, normally known as DNA vaccination, is discussed below, but is to a large extent an equivalent to mode d.

h) A live or viral vaccine comprising nucleic acids as discussed under g—also this mode is an equivalent to the modes in c and g, but as long as it will be possible to provide any of the modes a-c, this particular mode of the invention will be less preferred.

Typically, the immunization includes a primary immunization followed by at least one booster immunization. The immunogenic agent used in the primary immunization and in the at least one booster immunization may be identical or non-identical—non-identical formulations are already known from a large number of prophylactic vaccines, where the vaccine formulation used for priming is different from the booster formulation, cf. the polio vaccination scheme used in Denmark. It may also be interesting to prime with DNA or live vaccines and boost with polypeptide vaccines or fraction vaccines or vice versa.

It is expected that the therapeutic regimen may include periodic immunizations in order to maintain a high degree of immunological alertness in the vaccinated individuals. In such a regimen, the number of annual immunizations may amount to 1, 2, 3, 4, 5, 6, and 12. More specifically, 1-12 times per year is expected, such as 1, 2, 3, 4, 5, 6, 7, 8, 9, 10, 11, or 12 times a year to an individual in need thereof, since it has previously been observed that the memory immunity induced by the use of vaccines may not be permanent.

Due to genetic variation, different individuals may react with immune responses of varying strength to the same vaccine. Therefore, the vaccine according to the invention may comprise several different polypeptides (which is of course the case for modes a-c) in order to increase the immune response. The vaccine may therefore comprise two or more antigens derived from Leptospira.

### Preparation, Formulation and Administration of Immunogens

When effecting presentation of the immunogenic agent to the human immune system by means of administration thereof, the formulation of the immunogen follows the principles generally acknowledged in the art. Thus, for the formulation of any of the above-mentioned putative immunogens, the formulation will be selected by the person skilled in the art to best suit the type of immunogen chosen. Thus, the immunogenic agent generally comprises a pharmaceutically acceptable carrier, vehicle or diluent.

The vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines used in the invention are conventionally administered parenterally, by injection, for example, either subcutaneously, intracutaneously, intradermally, subdermally or intramuscularly. Additional formulations which are suitable for other modes of administration include suppositories and, in some cases, oral, buccal, sublinqual, intraperitoneal, intravaginal, anal, epidural, spinal, and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

For instance, preparation of vaccines which contain peptide sequences as active ingredients is generally well understood in the art, as exemplified by U.S. Pat. Nos. 4,608,251; 4,601,903; 4,599,231; 4,599,230; 4,596,792; and 4,578,770, all incorporated herein by reference.

One preferred embodiment of the invention utilises multiple presentations of B-lymphocyte epitopes derived from Leptospira. This effect can be achieved in various ways, e.g. by simply preparing fusion polypeptides comprising the structure (Leptospira polypeptide antigen)ₘ, where m is an integer ≧2 and then introduce the modifications discussed herein in at least one of the sequences. It is preferred that the modifications introduced includes at least one duplication of a B-lymphocyte epitope and/or the introduction of a hapten. These embodiments including multiple presentations of selected epitopes are especially preferred in situations where merely minor parts of a Leptospira polypeptide are useful as constituents in a vaccine agent.

Recombinant polypeptides from Leptospira are prepared according to methods well-known in the art. Longer polypeptides are normally prepared by means of recombinant gene technology including introduction of a nucleic acid sequence encoding polypeptide into a suitable vector, transformation of a suitable host cell with the vector, expression of the nucleic acid sequence, recovery of the expression product from the host cells or their culture supernatant, and subsequent purification and optional further modification, e.g. refolding or derivatization.

Shorter peptides are preferably prepared by means of the well-known techniques of solid- or liquid-phase peptide synthesis. However, recent advances in this technology has rendered possible the production of full-length polypeptides and proteins by these means, and therefore it is also within the scope of the present invention to prepare the long constructs by synthetic means.

For recombinant production, nucleic acids encoding relevant Leptospira proteins are necessary tools.

The nucleic acid fragments encoding Leptospira proteins will normally be inserted in suitable vectors to form cloning or expression vectors. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector. Preferred cloning and expression vectors are capable of autonomous replication, thereby enabling high copy-numbers for the purposes of high-level expression or high-level replication for subsequent cloning.

The general outline of a vector for use in the invention comprises the following features in the 5′→3′ direction and in operable linkage: a promoter for driving expression of the nucleic acid fragment encoding the Leptospira protein, optionally a nucleic acid sequence encoding a leader peptide enabling secretion (to the extracellular phase or, where applicable, into the periplasma) of or integration into the membrane of the Leptospira protein, the nucleic acid fragment encoding the Leptospira protein, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in humans (i.e. when using the vector in DNA vaccination) it is for security reasons preferred that the vector is not capable of being integrated in the host cell genome; typically, naked DNA or non-integrating viral vectors are used, the choices of which are well-known to the person skilled in the art.

The vectors of the invention are used to transform host cells to produce Leptospira protein. Such transformed cells can be cultured cells or cell lines used for propagation of the nucleic acid fragments and vectors, or used for recombinant production of Leptospira protein. Alternatively, the transformed cells can be suitable live vaccine strains wherein the nucleic acid fragment (one single or multiple copies) have been inserted so as to effect secretion or integration into the bacterial membrane or cell-wall of the Leptospira protein.

Preferred transformed cells for production of Leptospira protein of the invention are microorganisms such as bacteria (such as the species Escherichia [e.g. E. coli], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism such as a fungus, an insect cell, a plant cell, or a mammalian cell. Most preferred are cells derived from a human being, cf. the discussion of cell lines and vectors below.

For the purposes of cloning and/or optimised expression it is preferred that the transformed cell is capable of replicating the nucleic acid fragment of the invention. Cells expressing the nucleic fragment are preferred useful embodiments of the invention; they can be used for small-scale or large-scale preparation of protein or, in the case of non-pathogenic bacteria, as vaccine constituents in a live vaccine.

When producing protein by means of transformed cells, it is convenient, although far from essential, that the expression product is either exported out into the culture medium or carried on the surface of the transformed cell.

When an effective producer cell has been identified it is preferred, on the basis thereof, to establish a stable cell line which expresses a nucleic acid fragment encoding a relevant Leptospira expression product. Preferably, this stable cell line secretes or carries the ghrelin analogue of the invention, thereby facilitating purification thereof.

In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiae, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (5V40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

Preparation of live or attenuated vaccines will also follow the state of the art. For instance, vaccines for treatment of leptospirosis in dogs are already on the market and similar vaccines can be produced according to the same general principles, however utilising L. interrogans and a subunits derived therefrom.

When using polypeptides (such as Osp derived from Leptospira) they may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 5,000 μg (even though higher amounts in the 1-10 mg range are contemplated), such as in the range from about 0.5 μg to 2,000 μg or 0.5 μg to 1,000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

### DNA Vaccines

As an alternative to classic administration of a peptide-based vaccine, the technology of nucleic acid vaccination (also known as “nucleic acid immunisation”, “genetic immunisation”, and “gene immunisation”) offers a number of attractive features. This particular technology is embodied in variant g of the method of the invention.

First, in contrast to the traditional vaccine approach, nucleic acid vaccination does not require resource consuming large-scale production of the immunogenic protein. Furthermore, there is no need to device purification and refolding schemes for the protein. And finally, since nucleic acid vaccination relies on the biochemical apparatus of the vaccinated individual in order to produce the expression product of the nucleic acid introduced, the optimum posttranslational processing of the expression product is expected to occur; this is especially important since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is expected to be ensured by having the host producing the immunogen.

Hence, a preferred embodiment of the invention comprises effecting presentation of the Leptospira antigen(s) to the immune system by introducing nucleic acid(s) encoding the at least one Leptospira antigen into the animal's cells and thereby obtaining in vivo expression by the cells of the nucleic acid(s) introduced.

In this embodiment, the introduced nucleic acid is preferably DNA which can be in the form of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in a polymer, e.g. in PLGA (cf. the microencapsulation technology described in WO 98/31398) or in chitin or chitosan, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of other vaccines apply mutatis mutandis to their use in nucleic acid vaccination technology. The DNA may be cDNA encoding relevant Leptospira antigen such as an Osp, or synthetic DNA encoding epitopes derived from such a relevant protein.

As for routes of administration and administration schemes of vaccines which have been detailed above, these are also applicable for the nucleic acid vaccines of the invention and all discussions above pertaining to routes of administration and administration schemes for polypeptides apply mutatis mutandis to nucleic acids. To this should be added that nucleic acid vaccines can suitably be administered intraveneously and intraarterially. Furthermore, it is well-known in the art that nucleic acid vaccines can be administered by use of a so-called gene gun, and hence also this and equivalent modes of administration are regarded as part of the present invention. Finally, also the use of a VLN (cf. below) in the administration of nucleic acids has been reported to yield good results, and therefore this particular mode of administration is particularly preferred.

Furthermore, the nucleic acid(s) used as an immunization agent can contain regions encoding immunomodulating substances described herein as immunogenic carriers and coupling agents and fusion partners, e.g. the cytokines discussed as useful adjuvants. A preferred version of this embodiment encompasses having the coding region for the immunogenic Leptospira antigen and the coding region for the immunomodulator in different reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Under normal circumstances, the antigen-encoding nucleic acid is introduced in the form of a vector wherein expression is under control of a viral promoter. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

### Live Vaccines

A third alternative for effecting presentation of a Leptospira antigen to the immune system is the use of live vaccine technology. In live vaccination, presentation to the immune system is effected by administering a non-pathogenic microorganism which has been transformed with a nucleic acid fragment encoding relevant Leptospira protein or with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker P D, 1992, Vaccine 10: 977-990, both incorporated by reference herein. For details about the nucleic acid fragments and vectors used in such live vaccines, cf. the discussion below. It should be noted, though, that at pre-sent it is preferred to utilise a non-pathogenic Leptospira strain, because such

As an alternative to bacterial live vaccines, the nucleic acid fragment of the invention discussed below can be incorporated in a non-virulent viral vaccine vector such as a vaccinia strain or any other suitable pox virus.

Normally, the non-pathogenic microorganism or virus is administered only once to the animal, but in certain cases it may be necessary to administer the microorganism more than once in a lifetime in order to maintain protective immunity. It is even contemplated that immunization schemes as those detailed above for polypeptide vaccination will be useful when using live or virus vaccines.

Alternatively, live or virus vaccination is combined with previous or subsequent polypeptide and/or nucleic acid vaccination. For instance, it is possible to effect primary immunization with a live or virus vaccine followed by subsequent booster immunizations using the polypeptide or nucleic acid approach.

The microorganism or virus can be transformed with nucleic acid(s) containing regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitopes are produced as fusion partners to the immunomodulator. Alternatively, two distinct nucleotide fragments can be used as transforming agents. Of course, having the 1^{st} and/or 2^{nd} and/or 3^{rd} moieties in the same reading frame can provide as an expression product, an analogue of the invention, and such an embodiment is especially preferred according to the present invention.

### Other Immunogens of the Invention

As an alternative to the use of Leptospira derived antigens or nucleic acids, it is also possible to immunize by using anti-idiotypic antibodies or even mimotopes that mimic antigens derived from Leptospira. The technologies for preparing anti-idiotypic antibodies that mimic a Leptospira epitope are known in the art and entail provision of monoclonal anti-Leptospira antibody followed by subsequent production of antibody that binds the idiotype of said anti-Leptospira antibody. Mimotopes can be isolated from libraries of random peptides that are screened in phage display against antibodies that bind Leptospira antigen specifically.

### Enhancing Immune Responses

Some of antigens/immunogens are sufficiently immunogenic for a vaccine, but in many cases it will be of interest to enhance the immune response.

Since the early days of immunology, it has been known that not only the antigen is of importance when it is desired to confer immunity. Many vaccines and other immunogenic compositions (e.g. those used for raising antibodies for diagnostic purposes), include a number of other features of which the 2 most important are 1) immunogenic carriers and 2) adjuvants.

### Carriers and T_{H} Epitopes

Immunogenic carriers are normally polypeptides or proteins that are coupled chemically to the antigen in order to render it (more) immunogenic. During the last 30 years it has become apparent that immunogenic carrier proteins exert their effect because they include a large number of T_{H} epitopes that taken together ensure that the immunogen (including its carrier part) is capable of stimulating T_{H} lymphocytes, thereby facilitating activation and proliferation of cytotoxic T cells or proliferation of B-cells and subsequent antibody production.

Traditionally, such carrier molecules are conjugated via chemical linking to the antigen, thereby providing an antigen/carrier or hapten/carrier conjugate. Typical carrier proteins for this use are antigens that are universally recognized, i.e. such proteins as the traditional carrier molecules keyhole limpet hemocyanin (KLH), tetanus toxoid, diphtheria toxoid, and bovine serum albumin (BSA).

In recent years and after the emergence of recombinant gene technology, a refinement of this traditional approach has been used, where the carrier proteins have been fused to protein antigens so as to provide fusion proteins—this approach can take all forms, where the antigen is inserted in the carrier protein and vice versa, or where the carrier protein and the antigen have been fused end-to-end. In embodiments of the present invention where immunization with single protein antigens derived from Leptospira is contemplated, this approach is preferred, since the control over the immungen's properties is greatly enhanced.

An even more recent version of this approach utilises defined short T_{H} epitopes instead of complete carrier proteins. There exist a number of naturally occurring “promiscuous” (also known as universal) T-cell epitopes which are active in a large proportion of individuals of an animal species or an animal population.

The promiscuous epitope can according to the invention be a naturally occurring human T-cell epitope such as epitopes from tetanus toxoid (e.g. the P2 and P30 epitopes, cf. e.g. WO 00/20027), diphtheria toxoid, Influenza virus hemagluttinin (HA), and P. falciparum CS antigen.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in the analogues used according to the present invention. Cf. also the epitopes discussed in the following references which are hereby all incorporated by reference herein: WO 98/23635 (Frazer I H et al., assigned to The University of Queensland); Southwood S et al., 1998, J. Immunol. 160: 3363-3373; Sinigaglia F et al., 1988, Nature 336: 778-780; Chicz R M et al., 1993, J. Exp. Med. 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes which share common motifs with these.

Alternatively, the epitope can be any artificial T-cell epitope which is capable of binding a large proportion of MHC Class II molecules. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes in a protein antigen from Leptospira, which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA or an immunologically effective subsequence thereof. This, and other epitopes having the same lack of MHC restriction are preferred T-cell epitopes which could be present in an immunogen used according to the invention.

### Other Coupling and Fusion Agents

It can e.g. be of interest to target the immunogen to APCs or B-lymphocytes. This can be achieved by coupling to a specific binding partner for a B-lymphocyte specific surface antigen or for an APC specific surface antigen (or by fusing a nucleic acid immunogen to a nucleic acid sequence which encodes such a specific binding partner). Many such specific surface antigens are known in the art. For instance, the binding partner can be a carbohydrate for which there is a receptor on the B-lymphocyte or the APC (e.g. mannan or mannose). Also an antibody fragment which specifically recognizes a surface molecule on APCs or lymphocytes can be used (the surface molecule can e.g. be an FCγ receptor of macrophages and monocytes, such as FCγRI or, alternatively any other specific surface marker such as CD40 or CTLA-4). It should be noted that all these exemplary targeting molecules can be used as part of an adjuvant also, cf. below.

As an alternative or supplement to targeting the immunogen to a certain cell type in order to achieve an enhanced immune response, it is possible to increase the level of responsiveness of the immune system by coupling to a molecule that stimulates the immune system (and, again, by having nucleic acids encoding a protein immunogen being fused to nucleic acids that encode such a molecule). Typical examples are cytokines, and heat-shock proteins or molecular chaperones, as well as effective parts thereof. Suitable cytokines are those which will normally also function as adjuvants in a vaccine composition, i.e. for instance interferon γ (IFN-γ), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF); alternatively, the functional part of the cytokine molecule may suffice. With respect to the use of such cytokines as adjuvant substances, cf. the discussion below. Non-limiting examples of heat-shock proteins or molecular chaperones are HSP70 (heat shock protein 70), HSP90 (heat shock protein 90), HSC70 (heat shock cognate 70) GRP94 (also known as gp96, cf. Wearsch P A et al. 1998, Biochemistry 37: 5709-19), and CRT (calreticulin).

Other examples of immune stimulating molecules are toxins, such as listeriolycin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities.

Finally, the immunogen may include, together with or coupled to the antigen, a molecular moiety which enhances the presentation of the immunogen to the immune system (again, this mode of the invention may also be achieved when using nucleic acids encoding a protein antigen, where the molecular moiety is encoded by the nucleic acids). The art has shown several examples of this principle. For instance, it is known that the palmitoyl lipidation anchor in the Borrelia burgdorferi protein OspA can be utilised so as to provide self-adjuvating polypeptides (cf. e.g. WO 96/40718)—it seems that the lipidated proteins form up micelle-like structures with a core consisting of the lipidation anchor parts of the polypeptides and the remaining parts of the molecule protruding there from, resulting in multiple presentations of the antigenic determinants. Hence, the use of this and related approaches using different lipidation anchors (e.g. a myristyl group, a myristyl group, a farnesyl group, a geranyl-geranyl group, a GPI-anchor, and an N-acyl diglyceride group) are preferred embodiments of the invention, especially since the provision of such a lipidation anchor in a recombinantly produced protein is fairly straightforward and merely requires use of e.g. a naturally occurring signal sequence as a fusion partner.

Another possibility is use of the C3d fragment of complement factor C3 or C3 itself (cf. Dempsey et al., 1996, Science 271, 348-350 and Lou & Kohler, 1998, Nature Biotechnology 16, 458-462).

Another approach includes coupling of the antigen to an inert carrier backbone which allows for presentation of multiple identical antigenic determinants. This is achieved by covalent coupling of the immunogen to certain molecules and, when necessary, together with foreign T_{H} epitopes or other of the molecular immunity enhancers described above. As such inert carriers, polymers can be used, e.g. polyhydroxypolymers, notably carbohydrates such as dextran, cf. e.g. Lees A et al., 1994, Vaccine 12: 1160-1166; Lees A et al., 1990, J Immunol. 145: 3594-3600, but also mannose and mannan are useful alternatives. Integral membrane proteins from e.g. E. coli and other bacteria are also useful conjugation partners.

### Adjuvants

As a supplement or alternative to the use of the above-discussed carriers and coupling agents, the use of immunologic adjuvants is also very important.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

Non-limiting examples of suitable adjuvants are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. Furthermore, all the molecules that have been discussed above for stimulating the immune system, for targeting immunogens or for enhancing presentation to the immune system are in principle also useful as adjuvants according to the present invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins such as QuilA and QS21 are interesting as is RIBI. Further possibilities are monophosphoryl lipid A (MPL), the above mentioned C3 and C3d, and muramyl dipeptide (MDP). Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic agent, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcγ receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned above. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; and latex such as latex beads.

Yet another interesting way of modulating an immune response is to include the immunogen (optionally together with adjuvants and pharmaceutically acceptable carriers and vehicles) in a “virtual lymph node” (VLN), cf. WO 99/44583. The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant.

Microparticle formulation of vaccines has been shown in many cases to increase the immunogenicity of protein antigens and is therefore another preferred embodiment of the invention. Microparticles are made either as co-formulations of antigen with a polymer, a lipid, a carbohydrate or other molecules suitable for making the particles or the microparticles can be homogeneous particles consisting of only the immunogen itself.

Examples of polymer based microparticles are PLGA and PVP based particles (Gupta R K et al., 1998) where the polymer and the antigen are condensed into a solid particle. Lipid based particles can be made as micelles of the lipid (so-called liposomes) entrapping the antigen within the micelle (Pietrobon P J, 1995). Carbohydrate based particles are typically made of a suitable degradable carbohydrate such as starch or chitosan. The carbohydrate and the antigen are mixed and condensed into particles in a process similar to the one used for polymer particles (Kas H S et al., 1997).

Particles consisting only of the antigen can be made by various spraying and freeze-drying techniques. Especially suited for the purposes of the present invention is the super critical fluid technology that is used to make very uniform particles of controlled size (York P, 1999 & Shekunov B et al., 1999).

### Antibacterial Therapy

Knowing that the causative agent is a bacterium also opens for direct antibacterial treatment in order to treat or ameliorate MS. It is, however, not altogether unimportant to bear in mind that the expected lifecycle of Leptospira warrants that antibacterial treatment should be synchronized with the development of the disease.

However, in general the present invention also relates to a method for treating or ameliorating multiple sclerosis (MS) in a human subject suffering from MS, the method comprising administering a therapeutically effective amount of an antibiotic exhibiting bacteriotoxic or bacteriostatic effect on L. interrogans.

Any antibacterial principle known in the art is applicable in this aspect of the invention; it is however contemplated that one of the mechanisms that allows Leptospira to escape the human immune system is the capability of the spirochetes to form cysts that are inaccessible to the immune system. Recent research has shown that bacteria are capable of a primitive form of communication (in the form of interbacterial chemical signalling) and it is contemplated that cyst formation by Leptospira takes place as a consequence of such signalling when the bacteria are beginning to suffer under the infected person's immunological counter attack. Thus, it is preferred to utilise an antibiotic which is capable of interfering with signalling between bacteria.

However, otherwise it is possible to utilise virtually any antibacterial principle known, so the antibiotic is preferably selected from the group consisting of antibodies or fragments thereof, bacitracin, cephalosporins, cycloserine, penicillins, ristocetin, vancomycin, amphotericin B, colistin, imidazoles, nystatin, polymyxins, chloramphenicol, erythromycins, lincomycins, tetracyclines, aminoglycosides, nalidixic acid, novobiocin, pyrimethamine, rifampin, sulfonamides, trimetoprim.

The use of antibodies (passive immunization) deserves special attention since this does have a lot in common with the active immunization principles discussed above. Passive immunization with monoclonal antibodies would have the drawback that secondary immune mechanisms would not be activated, whereas administration of polyclonals would provide for the same secondary immunological effects as active immunisation.

At any rate, the antibiotic (be it a classical antibiotic or an antibody) should preferably be capable of entering the CNS from the vascular system in order to exert its effect where the Leptospira are active. The most preferred antibiotic is a tetracycline.

It is according to the invention advantageous to combine the antibiotic treatment with a further treatment regimen, which reduces or modulates the pathogenesis, said further treatment regimen being selected from the group consisting of an anti-inflammatory treatment regimen, treatment with a Leptospira toxin binding compound, treatment with a compound that inhibits Leptospira toxin production, and a compound that directly or indirectly promotes the degradation of Leptospira toxin. These embodiments aim at reducing the harmful effects of combating the Leptospira (both Leptospira toxins as well as local inflammation may contribute to a worsening of an MS attack).

In a preferred embodiment, the antibiotic (or antibody) is administered during or shortly after an MS attack. The rationale behind this is that the Leptospira are attacked shortly before they escape the immune system of the infected individual, i.e. in a state where they must be expected be most vulnerable to an antibiotic attack.

All the antibacterial therapies described above will be conventionally applied, i.e. taking into account the age, condition and weight of the patient as well as the fact that the infection is

### Diagnosing and Monitoring MS

The invention also provides for means of diagnosing and monitoring the progress of MS. The finding of L. interrogans derived material in a sample from a patient who is suspected of suffering from MS will provide a very good indication that said patient is indeed suffering from MS.

Any of a number of methods for determination of L. interrogans in a sample can be utilised. For instance, various immune assays (RIAs, ELISAs and other assays relying on the use of anti-L. interrogans antibodies as capturing agents) are useful. For example, capture of L. interrogans material with a fixed antibody followed by application of an antibody binding a different L. interrogans derived epitope is one option but also capture with a first antibody followed by capture of the first antibody with an antibody-binding antibody is a possibility. The skilled person will know how to device a suitable assay setup.

Alternatively, direct detection and/or quantification of L. interrogans in a sample can be accomplished by means of various molecular amplification assays such as PCR based assays. Nucleic acids in the sample that are specific for L. interrogans are specifically amplified by careful selection of L. interrogans specific primers which are used in a PCR procedure (or any other molecular amplification process capable of amplifying nucleic acids) and the presence of amplified material is subsequently be detected/quantified in an assay utilising an optionally labelled L. interrogans specific capture probe. The present inventors currently use the protocol described in Woo et al. 1997 (cf. the reference list).

Another method of the invention assesses the risk that a person is suffering from MS or will attract MS, the method comprising subjecting a sample obtained from the person to a test that determines the presence, in the sample, of antibodies generally reactive with Leptospira and antibodies specifically reactive with L. interrogans. The rationale behind such a test is that the presence of antibodies generally reactive with Leptospira will normally mean that the risk is decreased (due to protection from cross-reacting antibodies), whereas a demonstration that the sample comprises antibodies that are generally active with Leptospira and antibodies that are specifically reactive with L. interrogans is correlated with an increased risk—this is based on the assumption that the specifically reactive antibodies will only be present in the sample if the immunity conferred by the non-specific antibodies is insufficient to confer protective immunity against the L. interrogans.

Consequently, a demonstration of neither the presence of generally reactive antibodies or antibodies specific for L. interrogans is correlated with an average risk that the person suffers from or will attract MS.

Again, these assays can be performed in a standardized manner, using L. interrogans and Leptospira material as catching probes and subsequent visualization of the bound antibodies by methods generally acknowledged in the art.

A different approach is based on the finding that Leptospira in some patients are capable of escaping complement mediated lysis. It is assumed that it is such a Leptospira-patient interplay that will allow for the development of MS, so it is also possible to simply measure the person's ability to lyse a relevant Leptospira species with his serum (via the alternative complement pathway).

To increase the precision in the diagnostic methods described above, they may be performed in parallel (at least 2 of the assays), so that it becomes possible to consolidate the findings from each assay, meaning, of course, that several independent indications of increased risk of MS strengthens the assumption that the person is suffering from or is likely to attract MS.

It has surprisingly been found by the present inventor that one standard assay for determination of Leptospira antibodies, namely the indirect hemagglutination assay (IHA) is largely incapable of detecting Leptospira antibodies in MS patients, whereas such antibodies can be detected by use of e.g. an immunofluorescense assay (IFA), cf. the Example section. The reason seems to be that sera from MS patients include material (possibly antibodies) which is capable of blocking the agglutination reaction in the IHA, whereas such blocking components are not present in sera from patients suffering from Leptospirosis.

This opens for a very specific diagnosis of MS, namely a test comprising detection and/or quantification of Leptospira or antibodies against Leptospira (cf. above) combined with an IHA designed for Leptospirosis detection. If the IHA is negative, whereas the other test is positive, there is a high likelihood that the sample is from a patient suffering from or at risk of attracting MS.

In fact, the present finding is believed to be of more general applicability than merely for the purpose of diagnosing MS. The invention therefore also contemplates diagnostic assays aimed at an evaluation of risk-of-chronic-illness for specific antigens based on the inhibition of antigen-antibody reaction in hemaglutination assays, where the antibody is specific for the given antigen in the absence of sera from individuals defined as having increased risk of chronic illness. Sera from individuals are diluted and mixed with antibodies against the antigen. Subsequent inability of said antibodies to react with said antigen is interpreted as increased risk of chronic illness caused by the relevant pathogen. Further, the higher the dilution that allows the inhibition of antibody-antigen coupling, the higher the risk of chronic illness. The evaluation may be given exactly as outlined for leptospirosis causing risk-of-chronic-Leptospira interrogans related illness i.e., MS (as shown in example 3). For example: in diagnosing Lyme disease caused by Borrelia burgdorferi s.l., the Leptospiral antigen is replaced with Borrelia burgdorferi bacteria or Borrelia burgdorferi specific antigens. The Leptospiral antibodies are replaced with Borrelia burgdorferi specific antibodies. Sera from individuals which inhibit the antigen-antibody reaction will be regarded as those derived from patients having an increased risk of chronic Borrelia burgdorferi related illness. Precisely the same principle can be used for assessing the risk of chronic illness caused by other pathogens.

The above Leptospira-related methods may also be used to monitor the progress of MS in a patient. The method then requires several measurements using the same test in order to determine whether or not a difference is observed over time. This allows for 1) a simple overtime evaluation of the patient's disease status, but also for 2) an evaluation of the efficacy of an anti-MS treatment as described herein. Furthermore, the diagnostic method of the invention may be followed by a therapeutic regimen of the invention and vice versa.

### Kits and Pharmaceutical Packages of the Invention

Even though the means used in the therapeutic and diagnostic aspects of the invention may be known per se, their use for MS treatment, prophylaxis and diagnosis is believed by the present inventor to be novel.

Hence, packages containing any one of the therapeutic or prophylactic agents described above and (written) instructions for their use in therapy or prevention of MS are believed to be novel and inventive as are packages/kits for combined diagnosis/monitoring and treatment/prevention of MS.

### EXAMPLE 1

### Endemic Stability Studies in Search for Diseases with Unknown Etiology

Emerging and re-emerging diseases (such as MS) are receiving increasing interest in the scientific world. The concerns are based on the idea that diseases emerge due to new interaction between man and reservoirs of disease agents. While this may be true in many cases, this invention is based on the idea that what appears to be emergence of disease may in fact be a zoonosis mirroring endemic stability. Thus the emergence arises from an lack of contact to nature, rather than an increased contact.

Thus, the research underlying this finding is based on studies in endemic stability, where it is assumed that a disease in humans (in this case MS) which has no known etiological agent, is in fact a zoonotic disease which mirrors endemic stability in the reservoir host (in this case Rattus Norvegicus). It is believed that the present invention is the first to utilise studies of endemic stability in order to identify a pathological agent from a zoonosis as the etiological agent for a human.

The underlying reasoning that lead to the present invention is presented in the following and boils down to four basic discussions:

A) On the Probability that a Disease is Zoonotic

As will be evident from the following quotes, zoonotic diseases play a significant role in humans.

“There are 1415 species of endoparasitic infectious organisms known to be pathogenic in humans, many of which have only recently been recognized. These pathogens are responsible for an enormous global burden of disease and cause 14 million human deaths per year” (Woolhouse, 2002).

and

“Most human pathogens (868 species; 61% of the total) are zoonotic. The majority of these is associated with ungulate, carnivore and/or rodent reservoirs, although a substantial minority is associated with primates, bats, marine mammals, birds and other vertebrates” (Woolhouse, 2002).

“The Centers for Disease Control and Prevention's (CDC) acute infectious disease prevention and control strategies were largely developed from experiences with vaccine-preventable childhood diseases, sexually transmitted diseases, hepatitis, and other diseases for which traditional clinically based or laboratory-based surveillance can provide the base for intervention activities such as vaccination or antimicrobial chemotherapy. For the zoonoses and for diseases caused by species-jumping agents, prevention and control strategies have come from diverse bases.” (Murphy, 1998)

Aside from stating that there are more than even odds that a disease may be zoonotic, it also became clear for the present inventor that much of the attention in medical research historically has been given to diseases that are not zoonotic. The latter should lead to even better odds in current research. Both of the above-cited authors view the change in the pattern of disease as a consequence from new etiological agents or by new closer interactions between humans and nature. The latter is connected to “disturbance” of natural transmission cycles.

However, looking at western civilization it seems that just as many problems comes from absence of something rather than the presence of something new. Hence, when dealing with obesity we may easily conclude that in many cases, the cause is lack of physical exercise. Also in the case of allergies, absence of natural infection has been included as possible causes. But how would we explain increased disease a reduced force of infection?

### B) On Endemic Stability

“Endemic stability is an epidemiological state of a population, in which clinical disease is scarce despite high level of infection. The notion was developed to describe patterns of tick-borne disease in cattle. However, we propose a general model of endemic stability that is applicable to a broader range of diseases that are important in public health, including malaria, rubella, and mumps. We postulate that endemic stability requires only that (1) the probability, or severity, of clinical disease after infection increases with age, and (2) after one infection, the probability that subsequent infections result in disease is reduced” (Coleman and Woolhouse, 2001).

Endemic stability is usually understood to be related to the natural host of a disease which also is the case for Coleman and Woolhouse (2001), but since infectious agents may react and induce similar responses in many different species, the present inventor has concluded that it is more than likely that human epidemiology may mirror endemic stability in a non-human species. In other words, the disease would then be a zoonosis mirroring endemic stability and it will be expected that the description of the disease will be as shown in FIG. 1a.

### C) On Using Spatial, Temporal and Demographic Dimensions

It is the standard procedure to investigate epidemiology by using several dimensions. These may involve spatial, temporal and demographic data.

Spatial dimension is traditionally not the preferred dimension because it often requires that the data from different regions has to be pooled. Typically there will be unanswered questions as to the comparability of the data, which appears uninviting to the researcher. The temporal dimension is unaffected by such problems but follows a timeline in a certain space, and thus this type of study is one of the preferred tools. The demographic dimension contains all types of demographical classes: sex-ratio, risk-groups etc., and has been used to direct studies to areas of high risk i.e. exposure.

When assuming that a disease mirrors endemic stability in a different species, two of these dimensions may in part fail to assist in the search. Temporal dimensions fail because they usually assume that when exposure is high then incidence is high. Especially high peaks in exposure give a distinct drop in incidence and because statistics will weigh such periods heavily, statistics will reject the hypothesis of a correlation between the true etiological agent and the disease.

Demographic correlations may fail because sex-ratios become variable. Usually men are more exposed than women in zoonotic diseases, at least in western culture where men are more involved with agriculture and hunting. At low exposure, incidence may be high for men and low for women, but as exposure increase for both sexes the incidence decrease for men and increase for women. Knowing which sex is most heavily exposed requires that the source of infection is known (is it by handling meat, by working in the garden, or by performing other acts in specific environments?). The lack of knowledge of this point means that demographics such a sex-ratios are of little use. Also the age of onset may be affected. The difference in exposure between sexes can lead to a situation where men are being infected at young age and female at older age or vice versa.

The spatial dimension is, however, not likely to fail in the search for an etiological agent. Using mean prevalence over long periods in time on different scales should create convincing evidence. The use of islands is especially helpful in tracking down reservoirs because the wildlife will be reduced to fewer species. Unfortunately, human population densities may be low and medical services of a different character than in heavily populated area, which means that the analysis includes data of variable quality. Still, examinations of the spatial dimension to identify the reservoir become a cornerstone in the search for an ethological agent for a zoonosis mirroring endemic stability.

### D) Guidelines and Conclusions

It is not realistic to make any sound scientific conclusions based on spatial analysis alone, no matter how many different scales are used. The spatial scale is necessary to identify co-occurring reservoir hosts, but cannot carry the burden of evidence alone. Having identified the reservoir should give an opportunity to either (i) reconstruct other dimensions, making sense of incidence and sex-ratios or (ii) narrowing the field down to a possible ethological agent. In the latter case the possible ethological agent can be used for reconstructing other dimensions directly.

In brief, the ideal method has the following four parts, where the analysis will move stepwise from a-d or directly from a to c and d.
a) Primary exploration of spatial patterns—a search for the reservoir
b) Primary reconstruction of temporal and/or demographic dimensions
c) Secondary exploration part—a search for the etiological agent
d) Secondary reconstruction of temporal and/or demographic dimensions

It can furthermore be added that the approach is warranted when the disease displays:
Geographical gradients
Contradicting evidence in sex ratios in otherwise reliable data.
Stability in time mirroring carrying capacity and density dependence on some time-scale

Mirroring of endemic stability resembles the well-known catch effort relation used in consideration of sustainable harvest in applied ecology. Usually the catch effort relationship describes the amount of fish that will be caught at a given fleet size. Unlike many other ecological models the catch effort model is a static model demonstrating the equilibrium point for the system.

Low effort yields low harvest. As the effort increase the harvest increase, until the effect of the harvest reduces the reproduction of the stock. Increasing the effort further may cause the stock to collapse. Similarly, in zoonotic diseases mirroring endemic stability, few people get sick when few reservoir hosts are available. The prevalence of disease increases as the exposure increase. However, as the exposure increase many people get infected in their youth and do not develop clinical disease. At a very high exposure the disease collapses i.e., the whole population gets infected in their youth and no clinical cases are recognized.

Hence, in translating the catch effort model into epidemiology, catch resembles mean yearly incidence or prevalence of disease and effort resembles reservoir densities. Thus one can also describe the catch per unit effort (CPUE) or rather prevalence per unit exposure (PPUE) of the disease and plot this against reservoir densities to find a Maximum Sustainable Prevalence. When the model is correct, a linear relationship should be observed either directly of by log transformation (FIG. 1b). The result is that rare diseases can be described by prevalence, ideally over many years, which is less sensitive to temporal variations. It can also be predicted that, as in catch effort relations the efficiency of transmission (catch ability) may change in time, which may influence the results (FIG. 1b). For further details, cf. Sparre and Sibren, 1998.

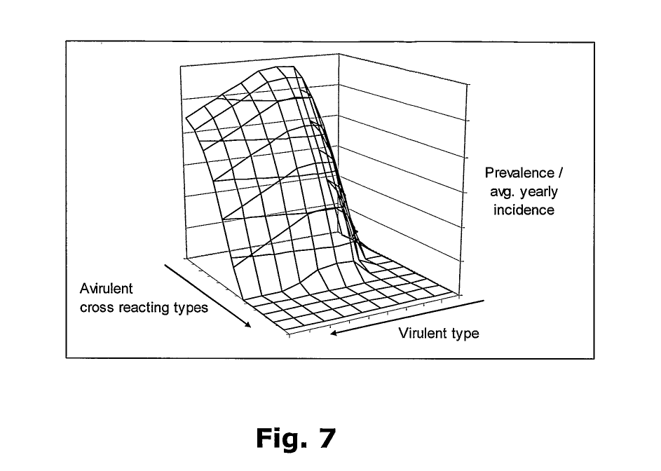

Furthermore special attention must be given to the possible interaction of other biological immunological cross reacting agents, as they may reduce the comparability between different areas. When no other biological immunological cross reacting agents exists, the relation between mean annual incidence or prevalence is as outlined in FIG. 1. As additional agents are added, which generate protection, the probability of disease is reduced proportionally to the ratio between avirulent and virulent agents (FIG. 7). Thus protective immunity derived by other routes may eliminate clinical disease even at the peak of maximum sustainable prevalence.

On the individual level, a given person may live in an area with low level of a virulent strain and high levels of avirulent cross-protective agents. If this person is relocated to an area with high prevalence of disease, the risk of attracting disease will be inversely correlated with the age of the person at the time of relocation. Thus it is quite difficult to determine the reason for low prevalence of disease because it can originate from three different causes: low presence of a virulent agent, high presence of a virulent agent, or high presence of cross-protective avirulent agents.

Migration studies for a given disease can assist in determining what areas fall within the same level of cross-protective avirulent agents. If there is no effect on risk of developing disease for any age group for any direction of movement between areas, they belong to the same mirror of endemic stability. If risk is reduced for older people alone, when moving to a new area, compared to the native population, the original location offers high levels of cross protective avirulent agents. Similarly, young people may transform to having low risk after a number of years in a low risk area. If a lower risk is not obtained after a number of years, a low risk status of that area is caused by absence of both virulent and avirulent agents.

Assembly of the whole pattern in FIG. 7 can thus be done by carefully evaluating the change in risk by monitoring migration of people between zones with well characterized risk.

In continuing this line of thought, the present inventor has found that it can be expected that incidence shows diminishing returns. Current year's incidence is likely to influence next years incidence as susceptible people are “harvested” or escapes from the population. High incidence this year is followed by low incidence next year. The plot is similar to plots of specific growth rate versus population size in a logistic model of population growth. For the endemically stable disease the change in incidence per incidence declines and becomes zero at the long term incidence of the population (carrying capacity, FIG. 1c). A straight forward linear relationship may be disrupted by changes in human demographics, behaviour of the reservoir host, changes in infectivity etc. It may thus be difficult to identify and interpret patterns.

Thus the present inventor's working definition of a mirror of endemic stability is only likely to be given as the relationship linear between log PPUE and exposure given e.g. as reservoir densities (FIG. 1b), because it is the most stable and well-tested (in fishery management) correlation.

### Conclusion

Even though exploration of disease patterns assuming endemic stability is believed to be a novel approach there is a substantial ecological basis for testing that the requirements are fulfilled. Thus, it can be tested and used in the pursuit of finding the cause for diseases with unknown etiological agent.

### EXAMPLE 2

### Search for a Zoonosis Likely to Cause MS

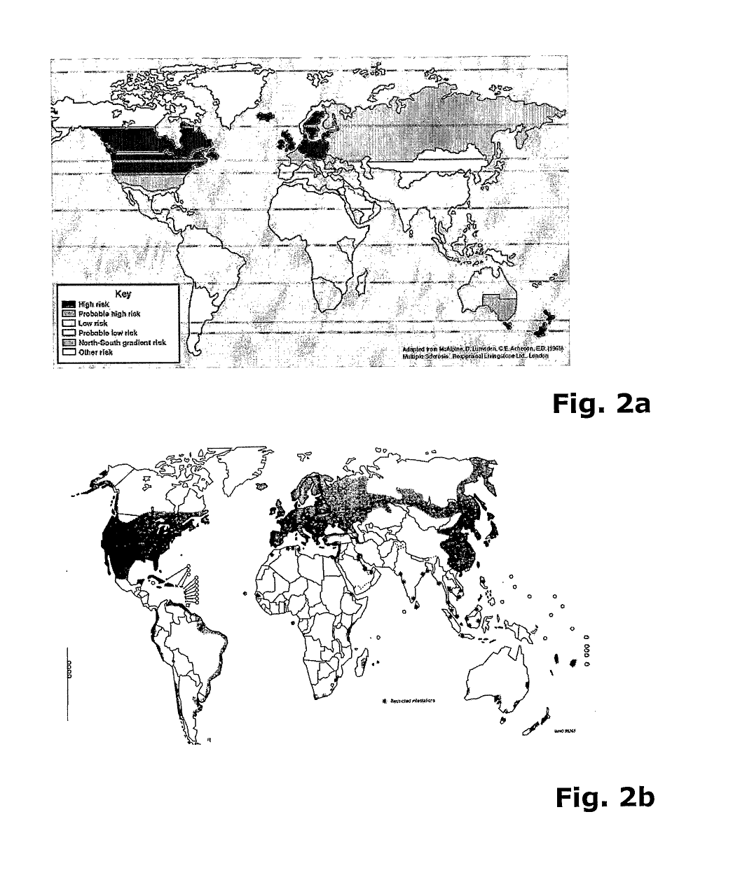

Multiple Sclerosis (MS) was chosen a preliminary test in a search for zoonoses mirroring endemic stability, due to the unclear epidemiology of the disease. The reports of gradient in e.g., Australia (Hammond et al. 2000) added to the probability that the analysis could bring forth evidence that would further the understanding of the disease. This also extends to the world-wide scale, where the pattern bares a promise of a special factor which has this unique distribution (FIG. 2a). On the surface it seems that the Eurasians have been at greater risk than other ethnic groups. Thus, when trying to attach the course of the disease to a possible reservoir all animals that are associated with the European culture wild and domestic are of interest.

Rather than dealing with all possible reservoirs investigated in the process, this article pre-sent what appears to be the most likely candidate based on spatial correlations. Thus, this paper addresses the distributions of Norwegian rat (Rattus norvegicus) and Leptospira as the etiological agent of MS.

### Materials and Methods

Exploring a zoonosis mirroring endemic stability has at this time no established methodology. Therefore the methodological issues have been developed addressed iteratively while performing this test case. As stated in Jensen (this issue) the process has lead to the conclusion that this type of study ideally includes a:
a) Primary exploration of spatial patterns—a search for the reservoir
b) Primary reconstruction of temporal and/or demographic dimensions
c) Secondary exploration part—a search for the etiological agent
d) Secondary reconstruction of temporal and/or demographic dimensions

The following section give the information regarding the four steps in the analysis. It should be noted that in the explorative parts, reports on rats were treated as a measure of contact. Each contact is assumed to correspond to a given probability of infection with etiological agents: Rat reports=Rat activity×Human activity=Cases of disease.

In the reconstructive parts rat rapport was adjusted for human activity giving a measure of rat activity: Rat activity/Human activity=Cases of disease/Human activity=Prevalence of disease.

The basic data on demographics, rat occurrence, multiple sclerosis (MS) and leptospirosis required to perform the analyses are given in Table 1.

### Primary Exploration of Spatial Patterns—a Search for the Reservoir

Local scale analysis was performed by on the reported occurrence of rats in 19 counties in Denmark (Miljøstyrelsen, Denmark) available on www.mst.dk and prevalence of MS in Denmark based on data from the Danish MS Register. The use of means over long period and prevalence data should ensure that epidemiological aspects such as extended periods between infection and MS debut did affect possible correlations.

Medium scale analysis was performed by analysing the occurrence on the Faeroe Islands as presented by (Kurtzke and Heltberg, 2001) and corresponding data for rats compiled by (Bloch, 1999). Since the distribution of R. norvegicus on the islands may have been different several decades ago, only the incidence rapports from the 3 and 4 epidemic on the islands were used.

World-wide distribution was given by maps of MS from (www.edlib.med.utah.edu/kw/ms/mml/ms_worldmap.html) and Norwegian rats (Gratz N. (unknown date)).

Primary Reconstruction of Temporal and/or Demographic Dimensions

Catch effort relationship for MS and rats was investigated by log transformed Prevalence Per Unit Exposure (PPUE) for a linear relationship with rat densities. Rat densities were estimated as rat observations divided by inhabitants in each county.

The core of the geographical distribution of R. norvegicus in Europe can be assigned to Southern Scandinavia. Based on the idea that densities of a given species may be greater in the core than at the edges of its distribution, the variation in prevalence of MS in Europe was analysed. Hence it was analysed whether MS is likely to be more frequent at latitude of 10° and longitude of 58°, based on recent data for MS in Europe (Roseti, 2001).

### Secondary Exploration Part—a Search for the Etiological Agent

Since R. norvegicus was identified as possible reservoir this part of the analysis examined the possibility that Leptospirosis was associated with MS. All diagnosis of Leptospirosis in Denmark is performed by Statens Serum Institut, Copenhagen, by agglutination test. The prevalence data was taken from recent records from 1980-2002 (Lemcke et al., in Press).

Secondary Reconstruction of Temporal and/or Demographic Dimensions

In making the assumption that MS is caused by Leptospira, it is also assumed that leptospirosis is mirroring endemic stability. Therefore Catch effort relationship (PPUE) for leptospirosis and rats was investigated as done for MS and rats in the primary reconstructive phase.

Finally the analysis included an analysis of the interaction between MS and leptospirosis. Because the diagnosis of leptospirosis is followed by antibiotic treatment eliminating the infection, it would be expected that counties with significantly higher focus on leptospirosis than average, would have lower overall incidence of MS. Residuals from the catch effort relationship for leptospirosis was taken as a measure of discovery. The ratio between MS and leptospirosis (stating the probability of leptospirosis turning into MS) and the estimated level of discovery for leptospirosis was examined.

In case that MS is indeed a zoonosis mirroring endemic stability, it can be expected that it displays density dependent fluctuations, similar to a population at its carrying capacity. The etiological agent uses humans as a “resource” and when depleted the incidence drops. Recruitment of susceptible humans is likely to vary though time and the carrying capacity will therefore be dynamic in time.

The initial description of the dynamics of MS in Denmark was performed by plotting the change in incidence against incidence ((MS incidence t−MS incidence t+1)/MS incidence)) for males, females and both sexes. This was used to identify different settings in the environment influencing the dynamics of the disease.

The characterisation of the endemic stability was illustrated by plotting yearly MS incidence versus rat densities. MS incidences originated from the Danish Multiple Sclerosis Register. Rat densities originate from requests for information on R. norvegicus to The Danish Pest infestation Laboratory (DPIL, Statens Skadedyrlaboratorium, 1965-1994). Rat densities were calculated as specific request on R. norvegicus divided by the total number of requests to DPIL. The data can only be taken as a representation of rat densities on the island of Zealand, due to the location of DPIL. The data is therefore not a necessarily a very good representation for the entire area of Denmark. Still no other data seem to cover the long period of 1965 to 1994. Data from 1981 was excluded from the analysis, under the assumptions that it is an error in the reports. The reports from this year is 472, while all other records lies around 100 to 200 (mean: 181 SD: 76).

In addition, male and female yearly incidence were plotted on separate x-axis, by multiplying rat densities with a factor k for males while keeping it equal to rat abundance for females. This procedure allowed the pattern for both sexes to overlap in the same graph. The factor was interpreted as a measure of differences in exposure. The final test was an examination of the variation in sex ratios at increasing rat densities.

### Statistical Analysis

The statistical analysis of the data is fairly limited. Most of the results are analyses by linear regression in a standard spreadsheet (Microsoft Excel). Two other types of analysis were performed in the primary phase of the evaluation.

Data on MS and rats on the Faeroe Island was analysed in a 2×3 frequency table. R. norvegicus was noted as present or absent. MS was noted as absent and present in one or two epidemics.

The analysis of MS prevalence in Europe included 29 countries. United Kingdom and Norway was divided into a northern and southern part each, due to numerous available data and an extended area. Longitudes and latitudes were assigned to each country based on what could be seen at the centre of the individual country. These records where then used to calculate the distance from a presumed optimum at 10° latitude and 58° longitude. In addition the prevalences were assigned to one year of publication, which in some cases meant that the prevalence was the average of several studies. The prevalence was analysed in a general linear model in the model given in equation 1 with a total of 69 observations.

MSprev=year dist to opt. longitude|dist to opt. latitude Equation 1

MS prev: The prevalence of MS per 100.000, Year: Year of publication, Dist to opt. longitude: Degrees departure from 58° longitude, Dist to opt. latitude: Degrees departure from 10° latitude.

Both analyses was performed in SAS 8.0 statistical software (SAS inc, NC, USA)

### Results

### Primary Exploration of Spatial Patterns—a Search for the Reservoir

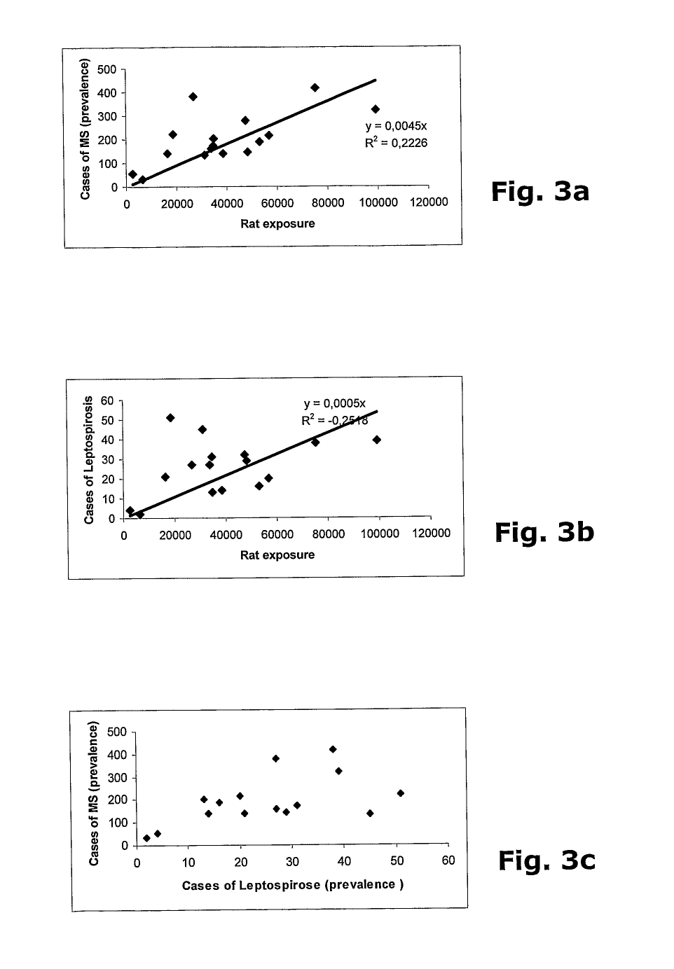

MS and Rats are found in all 19 counties in Denmark. A positive correlation between overall MS mean incidence and occurrence of R. norvegicus is noted (FIG. 3a).

MS is found on 5 of 17 islands on the Faeroe Islands, while rats is found on 7 islands (Table 2). Again the correlations seem to apply on an affirmative level but also reasonably on a negative level. The correlation was noted to be statistically significant (P<0.006).

The maps of world-wide distribution of MS and distribution of R. norvegicus seems to be concurrent (FIGS. 2a and 2b), or as concurrent as can be expected. For MS high incidences are seen in Europe, USA and Middle East. The occurrence of rats follows the same pattern and does not only correlate on an affirmative level but also on a negative level. In particular it should be noted that rats can account for MS in Iceland and Tasmania and for its absence in Greenland, Northern Australia, Africa and New Guinea.

Primary Reconstruction of Temporal and/or Demographic Dimensions

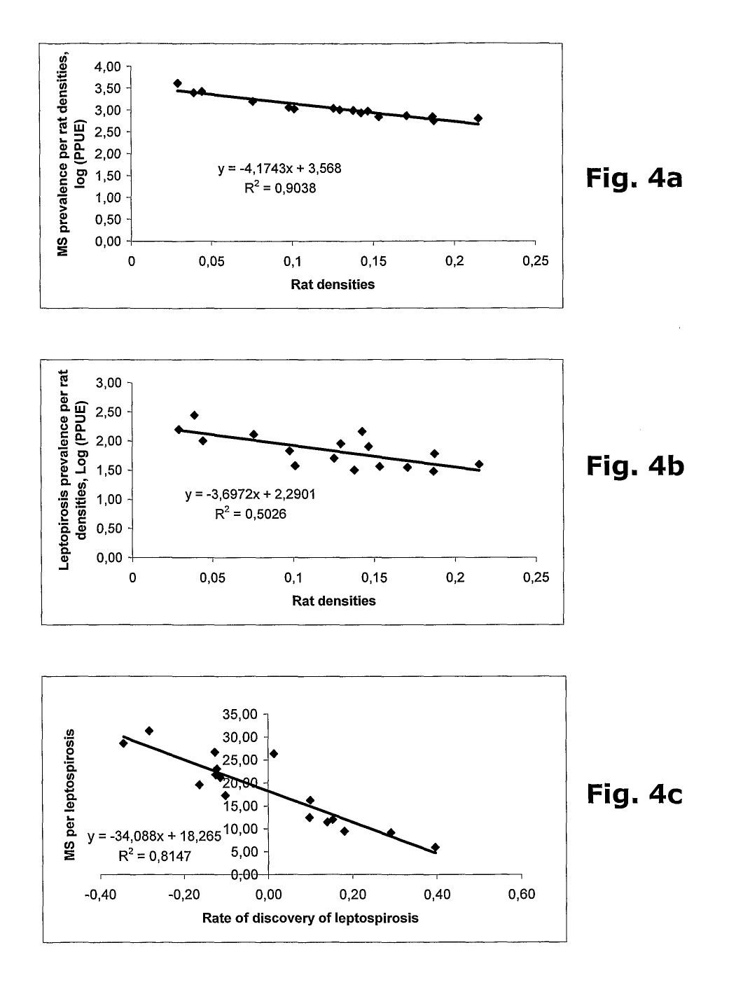

The plotted catch effort relationship for MS and rats give a quite good linear relationship (FIG. 4a, and Table 3), it thus fits within the working definition of a mirror of endemic stability.

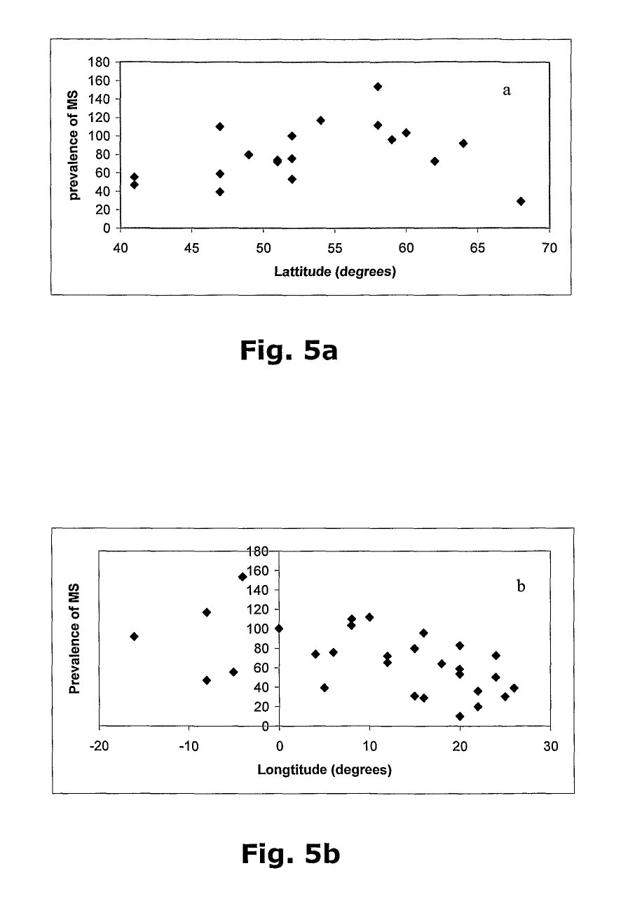

The core in the geographical distribution of rats in Europe was assigned to Southern Scandinavia. MS has distribution similar to this prediction, displaying a significant decline in MS prevalence's at latitudes greater and lesser than 10° (P<0.001, Table 4, FIG. 5a) and greater and lesser than longitudes of 58° (P<0.001, FIG. 5b). In additions it seems that there is an increase in MS prevalence during the years. The interaction between latitude and longitude suggest that there is a depression in MS at the core of its range.

### Secondary Exploration Part—a Search for the Etiological Agent

As stated, Leptospira infections can be among the possible etiological agents for MS. As for MS there seems to be a correlation between leptospirosis cases and exposure to rats (FIG. 3b). Two counties seem to differ from the rest: Ribe County and Copenhagen County, where a high number of leptospirosis cases are reported at a low level of rat abundance.

Secondary Reconstruction of Temporal and/or Demographic Dimensions

The plotted catch effort relationship for MS and rats give an acceptable linear relationship (FIG. 4b). Hence, leptospirosis can be described as mirroring endemic stability. Modelling PPUE for leptospirosis lead to residuals ranging from −0.34 to 0.40. Using this as a measure of relative rate of discovery for letospirosis provides a good linear correlation with the ratio MS/leptospirosis (FIG. 4c).

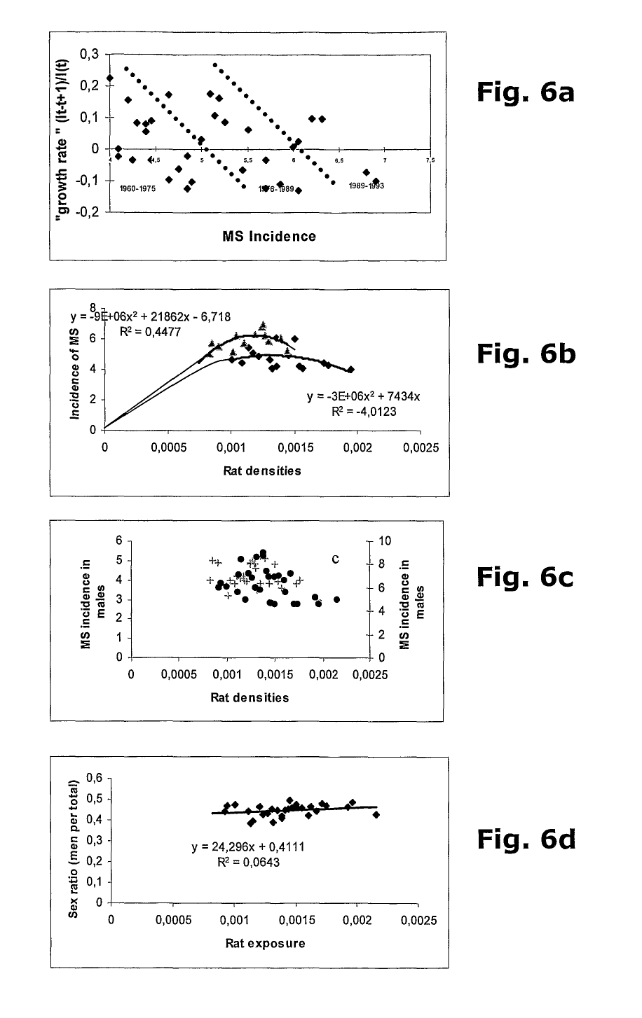

The plot of variation in incidence at each level of incidence shows increase at low incidence and drop in incidence at high incidence. In the period 1960-1975, the incidence level oscillated around an incidence of 4.5, but it moved on to oscillate around an incidence of 5.5 in 1976-1989. From 1989 to 1993 the oscillation occurred around and incidence of 6.5 (FIG. 6a).

Plotting the incidence for all cases does not give a clear bell shaped curve (FIG. 6b). Rather, it seems that there are two different peaks in incidence, corresponding to the two oscillation points at 4.5 and 6. Still it is possible to identify bell shaped curves in the pattern.

Separating male and females and adding them to the same curves provide a clearer picture of the bell shape, but the line is not apparent as the point fill out the peak in incidence (FIG. 6c). Creating the overlap requires that each sex is plotted on separate x-axis where rat densities for men is set to 90% of that experience by women. It is also clear, viewing the two independently defined y-axis' that the two sexes have different levels of incidence which may be explained by differences in the level of discovery of MS. Finally, the change in sex-ratios is not distinct (FIG. 6d), but some variation seems notable (FIG. 6d).

### Discussion

### Primary Exploration of Spatial Patterns—a Search for the Reservoir

The correlation between reports of rat infestations to the Danish municipal authorities seems to correlate quite well with the prevalence of MS in the 19 counties. The likelihood of this phenomenon occurring by chance is difficult to assess. Other animals may have a similar distribution as rats in Denmark e.g., mice, simply due to similar dependences on primary production.

In the Faeroe Island MS seems to follow the rat populations closely. It is quite surprising because the human population on some rat infested islands is quite small. The same is true for islands that are not infested with rats and therefore the interpretation must be cautious. Again the evidence is weak also because other diseases fall into similar although not identical patterns of the Faeroe Islands e.g., Parkinsons Disease (Wermuth et al. 1997). Still the strong statistical significance supports the idea that MS is associated with rats.

The correlation seen on the World-wide scale must be taken with some caution. In general it must be suspected that large scale evaluation has limited value, because the integrity of the data for the diagnosis of MS and the identifications of R. norvegicus may be compromised and not updated to the same period in time. Still it is reassuring to note that correlations exist even on a world wide scale. However, the evaluation of medium and local scale carry the greater burden of evidence in this case.

The three different scales each represent weak evidence which when tied together build an acceptable basis for further deductions. The concern of other mammals in Denmark with similar abundance variations are taken care of by the other correlations. Thus, there are only three mammals on the Faeroe Island (Bloch, 1999). Similar arguments are valid for the other correlations on a world-wide scale. The results therefore points to a close association between R. norvegicus and MS.

Primary Reconstruction of Temporal and/or Demographic Dimensions

The catch effort relationship for MS versus rats seems quite clear. Although plotting prevalence versus densities of rat does not produce anything recognizable as a bell-shaped curve, the plot of PPUE versus rat densities is quite clear. The reason that other plots are not producing clear correlation is that rat and human populations are quite stable and only describe a very small fraction of the full pattern. The same problems have been encountered in fishery management and are not surprising (Sparre and Sibren, 1998). Thus the analysis of MS though the period 1965 to 1994 for the whole of Denmark gave clearer results.

The idea of denser populations at the core of a species geographical distribution can be considered a rule of thumb, rather than an ecological fact. It is based on simple ideas concerning the ecological niche in most introductory chapters in all basic ecology book e.g., Smith and Smith (2002). It may relate directly to the densities of the populations due to a lower quality of habitats, but usually we expect that a rarity of suitable habitats is an equally likely cause for reduced densities. When MS prevalence's thus correlates to a possible core-effect in the R. norvegicus population, it can be seen following our expectations, in irrespective of the underlying cause. Thus it seems that the ecology of rats can be translated into the prevalence of MS in Europe in a similar manner as the simple linear relation ship between rats and MS in Denmark.

### Primary Conclusion

Assuming that the Brown Rat carries the etiological agent, a number of candidates can be suggested. Thus Leptospirosis is one of the better known infectious diseases carried by R. norvegicus (Planck and Dean, 2000). The disease is caused by Leptospira interrogans, which occurs in a number of different serovars. Leptospirosis pathology is primarily known for including renal failure, but studies have also shown that eye lesions are can be found in 85% of patients with confirmed leptospirosis (Martins et al., 1998). Since eye lesions are common in the preliminary phase of MS, and share a seasonal periodicity similar to MS onsets (Ya-ping, 2000), this may indicate that Leptospira is involved or associated with the cause of MS. The possible relation to spirochetal organisms has been suggested (Brorson et al., 2001) and a suggestion of MS being caused by Leptospira seems prudent. A full discussion of the possibilities of spirochetes being associated with MS is given by Marshall (1988)

More importantly MS and leptospirosis share as a clinical feature that incidence in children is low. Thus in both MS and leptospirosis the age of 15 years seems to be considered as a threshold for the risk of clinical manifestations (Hannond et al., 2000; Planck and Dean, 2000).

### Secondary Exploration Part—a Search for the Etiological Agent

Like MS, leptospirosis seems to correlate with rat densities. It could be seen as surprising that the correlation seems poorer than for MS, but several factors may play a role. Leptospirosis is caused by Leptospira interogans serovar icterhemoragia from rats. However, leptospirosis by serovar icterhemoragie only constitutes 26% of the total leptospirosis cases in Denmark, while e.g., serovar sejroe constitutes 22% (Lemcke et al., in press). Serovar Sejroe is associated with mice and the variations in mouse populations can therefore create deviation from the expected. Similarly Lemcke (in press) states that differences arise from variation in occupations, where land based aquaculture represent a high risk occupation. Most importantly the cause may be linked to marked differences in the focus on leptospirosis in the local hospital.

The effect that diagnosis and treatment of leptospirosis appear to have on the assumed risk that Leptospira infection manifesting itself as MS (FIG. 4c) is surprising. The chance of this correlation appearing incidental must be quite low. Knowing that leptospirosis can be caused by many different Leptospira serovar might point to the possibility that many types of Leptospira may diminish the risk of MS due to cross-reactivity between antibodies. This would be a quite simple explanation on how a high exposure is obtained in the profile of the mirror of endemic stability.

Secondary Reconstruction of Temporal and/or Demographic Dimensions

Interpreting the human population as the resource base of the disease is unconventional but rather simple. In doing so we assume that MS cases (the population) is recruited by newly susceptible people. Describing the growth rates of the disease as a function of its population size is a normal procedure. Unfortunately the susceptible population varies. In assuming that the disease mirrors endemic stability it is also assumed that young people are not susceptible. As the demographics of the human population change, the true prevalence of susceptible people changes.

The comparison of males and females suggests that men and women are almost equally exposed, and at the same time that the prevalence is greater for woman than for men. It represents a paradox that can partly be explained by differences in discovery. Thus the analysis postulates that discovery of MS in men and woman has a ratio of 6:4 (FIG. 6c). To test the first postulate the contact to Danish ophthalmologists by men and women in Denmark was extracted. From 1992 to 1996 the number of contacts has been reasonably stable around 370,000 for men and 580,000 for woman (Danmarks statistik, Danmarks Statistik-bank). It would therefore seem that the exposure is fairly even for men and woman, which may explain why there is no great variation in sex-ratios at considerable variation in the level of exposure.

A side effect of the differences in exposure means that the incidence in woman is still increasing while is has been stabilizing in men (Brønnum et al.). The interpretation in this scenario is that woman is still exposed beyond the MSI, and while the exposure is reduced due to changes in hygiene standard the Incidence is increased. Men being less exposed are experiencing less exposure and have cleared the top at MSI. To these quite complex interactions, the focus on leptospirosis is higher for men (Lemcke, in press) which also should contribute to the differences noted between sexes. This contribution is however small due to the overall focus on the disease in Denmark.

### Secondary Conclusion

The tight correlation between rats and MS on four different scales: in Denmark, on the Faroe Islands, In Europe and worldwide leads to the conclusion that it is highly unlikely that the etiological agent responsible for MS is not associated with rats. It can furthermore be added that it has to be a prevalent microorganism because MS is found in all locations were R. norvegicus is found. The Faeroe Islands constitutes a special case, because MS was unknown before the Second World War. Possibly the rat population was founded by a handful of rats, like it's known to be the case for the hare (Lepus timidus) population on the islands (Bloch, 1999). Presumably, the etiological agent was not present in this small number of individuals. During the British occupation of the island, rats are likely to have been imported in higher numbers, thus introducing the disease. These considerations suggest that the etiological agent should have a high but not too high prevalence in rat populations. Prevalences of Leptospira is found to be typically around 30% (10-70%) (Sunbul et al., 2001; Webster et al., 1995; Lilenbaum et al., 1993; Pereira and Andrade, 1988), which makes it possible for a handful of rats to invade an area without carrying Leptospira to be a possible, albeit rare, phenomenon.

In addition to a high prevalence of Leptospira in rats, mirrors of endemic stability as described here can only manifest it self, when prevalence in humans under ‘natural’ conditions is high. Since the reports of prevalence of 25-72% are found in China (Pan and He, 1995) and 25-35% in the slums of Rio de Janiero, Brazil (Pereira and Andrade, 1990), this last premise for the endemic stability is accounted for.

It is also important to emphasize that the correlations does not extent to closely related species like R. Rattus, because the areas of the world where R. rattus dominates (India and Africa) have very low prevalence of MS. Because the etiological agent is highly prevalent and rats are well-investigated animals used for many purposes in the medical society, it seems unlikely that the etiological agent is unknown.

Possibly Steiner was the first to establish that there was a close association between a spirochete and MS (Blackman and Putnam, 1936). Steiner described both the spirochetes and granular structures which was associated with the bacteria. He later described these as cysts from of the spirochete (Steiner, 1952). More importantly, Hassain and Diamond (1939) stated that these spirochetes can be found in all MS patients, even though it was only demonstrated in 8 of 8 possible cases. Leptospira, like the rest of the spirochetes are not normally associated with cyst formation. Still a compilation of known literature on the subject gives 200 articles published on the subject (Anonymous, 200?). One of these is a record of cyst formation in Leptospira (Inada et al. 1916). Adding to this, that Leptospira are the only spirochete to require long chain fatty acids to grow (Brock et al., 1994) gives a plausible explanation for its association with nerve tissue.

However, Steiner, Hassain and Diamond seemed convinced that the MS etiological agent and Weils disease etiological agent (Leptospira) were two separate organisms (Hassain and Diamond, 1939). But considering the effect that treatment of leptospirosis has had in Denmark (FIG. 5d), it is likely that they have cross-reacting antibodies, and are therefore likely to belong to the same group. Possibly, the effect arises from screening all sera using Leptospira serovar patoc at Statens Serum Institute. This serovar carries the antigens of the family Leptospira (Lemcke et al, in press).

Irrespective of the latter uncertainty, MS seems to fit the description of a zoonotic disease mirroring endemic stability. Concurrence with other possible types of epidemiology has not been tested, but since all pieces in the puzzle ties in to a single clear description it seems difficult to assume that there exist another type of epidemiology which can give a better description.

### Recommendation

The description provided above does not automatically lead to recommendations for rat control. These considerations must be given under the consideration of endemic stability and in fact increase of rat population may give better long term results than reduced populations. It depends on what the current level of infection is.

Each country has to investigate local epidemiology and plan on well-founded investigations. In Denmark it seems that any change in rat population is for the better, but most importantly the exposure has to be increased if control of MS has to rest on natural infection.

The alternative is vaccination against MS. Because MS is an endemic, stable disease we can assume with some certainty that the etiological agent is antigenically stable. The phenomenon could not exist without considerable protection after early infection in life. It is also likely that cross reactivity between Leptospira strains can give full or partial protection against the type (presumably L. interrogans) that causes MS.

It seems prudent under the precautionary principle as interpreted by Kaiser (1997) to call for:
More attention to diagnosis of leptospirosis and treatment of these cases. Attention should be given to treat the infection under the assumption that bacteria has penetrated into the spinal fluid.
Development of vaccines against MS. To our knowledge, there is no vaccine developed for Leptospira in humans. Since vaccines are produced for animals, development of a vaccine should be manageable within a short period of time.
We currently advise AGAINST the treatment of MS patients in remission. This has to await toxicological evaluation and investigation into the possible effect of toxins in the pathogenesis of MS. These investigations have now begun at Statens Serum Institute, Denmark.

### Conclusion

In summing up, it can be stated that the increasing prevalence of MS is not likely to arise from changes in nature. It is not a new thing emerging, rather it is human civilization withdrawing from its natural habitat, that causes the emergence. The importance of infections early in life and possibly partial protection from other infections emphasizes the importance of obtaining more information of the relationship between health and biodiversity.

TABLE 1
Background data on demographic, rat occurrence, MS and Leptospirosis in 19 counties in Denmark.

| Counties and | Demographic data | | | | | Multiple Sclerosis | | |
|---|---|---|---|---|---|---|---|---|
| municipals | | Area | Population | Data on R. norvegicus | | Prevalence | | |
| Country | Inhabitant | (ha) | density | Rat exposure | Rat densities | Male | Female | AVG |
| | 478405.524 | 8825 | 54.21 | 18603 | 0.038885 | 63.80 | 130.70 | 97.25 |
| Copenhagen | 87756.6667 | 877 | 100.06 | 2540 | 0.028944 | 101.10 | 139.20 | 120.15 |
| municipal | | | | | | | | |
| Frederiksberg | 610408.048 | 52595 | 11.61 | 26906 | 0.044079 | 87.00 | 149.20 | 118.10 |
| municipal | | | | | | | | |
| Frederiksborg | 343690.095 | 134742 | 2.55 | 34799 | 0.101251 | 78.40 | 137.50 | 107.95 |
| Roskilde | 216789.238 | 89148 | 2.43 | 16382 | 0.075566 | 92.60 | 147.40 | 120.00 |
| VestSjaeland | 284496.762 | 298377 | 0.95 | 53129 | 0.186747 | 94.90 | 164.70 | 129.80 |
| Storstroems | 257889.571 | 339802 | 0.76 | 48363 | 0.187534 | 76.00 | 135.50 | 105.75 |
| Bornholm | 46056.9048 | 58837 | 0.78 | 6339 | 0.137634 | 80.10 | 191.70 | 135.90 |
| Fyns | 461446.571 | 348584 | 1.32 | 99185 | 0.214944 | 102.40 | 170.70 | 136.55 |
| Sonderjutland | 251192.81 | 393834 | 0.64 | 38544 | 0.153444 | 86.50 | 131.80 | 109.15 |
| Ribe | 218824 | 313161 | 0.70 | 31180 | 0.142489 | 100.50 | 144.70 | 122.60 |
| Vejle | 333185.286 | 299664 | 1.11 | 56882 | 0.170722 | 93.40 | 161.20 | 127.30 |
| Ringkoebing | 267613.286 | 485348 | 0.55 | 34639 | 0.129437 | 94.00 | 170.00 | 132.00 |
| Aarhus | 601465.238 | 456073 | 1.32 | 75392 | 0.125347 | 104.20 | 171.10 | 137.65 |
| Viborg | 231167.286 | 412248 | 0.56 | 33867 | 0.146504 | 95.90 | 183.10 | 139.50 |
| North Jutland | 486117.143 | 617326 | 0.79 | 47606 | 0.097931 | 89.90 | 137.80 | 113.85 |
| Total or AVG | 5176504.43 | 4309441 | 11.27 | 624356 | 55390.91 | 90.04 | 154.14 | 122.09 |

| Counties and | | | | Leptospirosis | | |
|---|---|---|---|---|---|---|
| municipals | Cases | | | Prevalence Cases | | |
| Country | Sex-ratio | Male | Female | AVG | AVG | AVG |
| | 0.33 | 136 | 307 | 221.50 | 10.66 | 51 |
| Copenhagen | 0.42 | 41 | 68 | 54.50 | 4.56 | 4 |
| municipal | | | | | | |
| Frederiksberg | 0.37 | 265 | 497 | 381.00 | 4.42 | 27 |
| municipal | | | | | | |
| Frederiksborg | 0.36 | 144 | 262 | 203.00 | 3.78 | 13 |
| Roskilde | 0.39 | 105 | 175 | 140.00 | 9.69 | 21 |
| VestSjaeland | 0.37 | 139 | 240 | 189.50 | 5.62 | 16 |
| Storstroems | 0.36 | 103 | 189 | 146.00 | 11.25 | 29 |
| Bornholm | 0.29 | 19 | 45 | 32.00 | 4.34 | 2 |
| Fyns | 0.37 | 235 | 413 | 324.00 | 8.45 | 39 |
| Sonderjutland | 0.40 | 108 | 170 | 139.00 | 5.57 | 14 |
| Ribe | 0.41 | 109 | 158 | 133.50 | 20.56 | 45 |
| Vejle | 0.37 | 155 | 278 | 216.50 | 6.00 | 20 |
| Ringkoebing | 0.36 | 122 | 225 | 173.50 | 11.58 | 31 |
| Aarhus | 0.38 | 305 | 527 | 416.00 | 6.32 | 38 |
| Viborg | 0.34 | 110 | 211 | 160.50 | 11.68 | 27 |
| North Jutland | 0.39 | 217 | 341 | 279.00 | 6.58 | 32 |
| Total or AVG | 0.37 | 144.56 | 256.63 | 3209.50 | 8.19 | 409.00 |

TABLE 2
Presence and absence of rats and MS on 17 Faeoe Islands

| Island | R. norvegicus | 3. epidemic | 4. epidemic | MS 3 + 4 |
|---|---|---|---|---|
| Mykines | 0 | 0 | 0 | 0 |
| Vágar | 1 | 0 | 1 | 1 |
| Streymoy | 1 | 1 | 1 | 2 |
| Eysturoy | 1 | 1 | 1 | 2 |
| Kalsoy | 0 | 0 | 0 | 0 |
| Kunoy | 1 | 0 | 0 | 0 |
| Bordoy | 1 | 1 | 1 | 2 |
| Vidoy | 1 | 0 | 0 | 0 |
| Svinoy | 0 | 0 | 0 | 0 |
| Fugloy | 0 | 0 | 0 | 0 |
| Koltur | 0 | 0 | 0 | 0 |
| Hestur | 0 | 0 | 0 | 0 |
| Noelsoy | 0 | 0 | 0 | 0 |
| Sandoy | 0 | 0 | 0 | 0 |
| Skuvoy | 0 | 0 | 0 | 0 |
| Stóra Dimun | 0 | 0 | 0 | 0 |
| Suduroy | 1 | 1 | 1 | 2 |
| Total | 7 | 4 | 5 | 1, 4 |

1: MS or rats present, 0: rats or MS absent Information on MS: Kurtzke and Heltberg (2001) Information on rats: Bloch (1999) 2 × 3 frequency table including rat (present abscent) and MS abscent, present in one or two epidemics give: Chissq = 10.1, Df = 2, P = 0.006.

TABLE 3
Estimates of Prevalence Per Unit Exposure (PPUE) for MS and leptospirosis.

| | | | | | Probability of | | |
|---|---|---|---|---|---|---|---|
| | | | | | Leptospirosis | | |
| | MS per | | Leptospira per | | developing | | |
| | R. norvegicus | | R. norvegicus | | into MS | LPPUE | Residuals |
| | density | | density | | MS prevalence/ | Model | Relative Rate |
| Counties and | | Log | Leptospirosis | Log | leptospirosis | Log | of discovery of |
| municipals | MS PPUE | MSPPUE | PPUE | LPPUE | prevalence | LPPUE | leptospirosis |
| Copenhagen municipal | 2500.94 | 3.40 | 274.15 | 2.44 | 9.12 | 2.15 | 0.29 |
| Frederiksberg | 4151.17 | 3.62 | 157.48 | 2.20 | 26.36 | 2.18 | 0.01 |
| municipal | | | | | | | |
| Copenhagen | 2679.30 | 3.43 | 100.35 | 2.00 | 26.70 | 2.13 | −0.13 |
| Frederiksborg | 1066.16 | 3.03 | 37.36 | 1.57 | 28.54 | 1.92 | −0.34 |
| Roskilde | 1588.01 | 3.20 | 128.19 | 2.11 | 12.39 | 2.01 | 0.10 |
| VestSjaeland | 695.06 | 2.84 | 30.12 | 1.48 | 23.08 | 1.60 | −0.12 |
| Storstroems | 563.90 | 2.75 | 59.96 | 1.78 | 9.40 | 1.60 | 0.18 |
| Bornholm | 987.40 | 2.99 | 31.55 | 1.50 | 31.30 | 1.78 | −0.28 |
| Fyns | 635.28 | 2.80 | 39.32 | 1.59 | 16.16 | 1.50 | 0.10 |
| Sonderjutland | 711.33 | 2.85 | 36.32 | 1.56 | 19.58 | 1.72 | −0.16 |
| Ribe | 860.42 | 2.93 | 144.32 | 2.16 | 5.96 | 1.76 | 0.40 |
| Vejle | 745.66 | 2.87 | 35.16 | 1.55 | 21.21 | 1.66 | −0.11 |
| Ringkoebing | 1019.80 | 3.01 | 89.49 | 1.95 | 11.40 | 1.81 | 0.14 |
| Aarhus | 1098.15 | 3.04 | 50.40 | 1.70 | 21.79 | 1.83 | −0.12 |
| Viborg | 952.19 | 2.98 | 79.72 | 1.90 | 11.94 | 1.75 | 0.15 |
| NorthJutland | 1162.55 | 3.07 | 67.22 | 1.83 | 17.30 | 1.93 | −0.10 |
| Sum or AVG | 1338.58 | 3.05 | 85.07 | 1.83 | 18.26 | 1.83 | 0.00 |

TABLE 4
Result from the general linear model analysing geographical variation
in MS prevalence in Europe.

| Parameter | Estimate | SE | t Value | Pr > t |
|---|---|---|---|---|
| Intercept | −5572 | 934 | −3.8 | 0.003 |
| Year | 1.8 | 0.47 | 4.0 | 0.002 |
| Longitude | −2.9 | 0.51 | −5.7 | 0.001 |
| Latitude | −7.7 | 1.01 | −7.6 | 0.001 |
| Longt × Latt | 0.19 | 0.005 | 3.9 | 0.002 |

Rsq: 0.64

### EXAMPLE 3

### Detection of Anti-Leptospira Antibodies in Human Sera from MS Patients

### Materials and Methods

Sera were obtained from 20 MS patients from the Human Brain and Spinal Fluid Resource Centre, VA West Los Angeles Healthcare Centre, Los Angeles, Calif. 90073, which is sponsored by NINDS/NIMH, National Multiple Sclerosis Society, and Department of Veterans Affairs. Five samples each were received from progressive MS, progressive MS with relapses, relapsing-remitting MS in relapse and relapsing-remitting in remission. Information on sex and age was provided. Serological analysis was performed to detect antibodies from L. interrogans, Seoul Virus, and B. burgdorferi sl.

L. interrogans antibodies were detected using IFA. The sera were initially diluted 1:40 for screening and later 1:80, 1:160 and 1:320. Diluted antigen of L. interrogans serovar Patoc and Ictehemoragie was fixed on slides and ten μl diluted sample applied. The samples were then incubated for 30 min at 40° C. After rinsing twice with distilled water secondary anti-bodies (Polyclonal Rabbit-antihuman, IgA, IgG and IgM, DakoCytomation Denmark AS, LOT no: 00010719) were applied and incubated for 30 min at 40° C. After a final rinsing process, fluorescent light emission was gassed. The emission was graded on levels negative (n), borderline (b), positive (1+ to 4+). Samples diluted 1:80 receiving or exceeding a score of 1+ were accepted as being positive for antibodies. Samples that were negative at 1:40 dilution were regarded as undoubtedly negative.

L. interrogans strains and positive controls were kindly supplied by K. Krogfeldt, Statens Serum Institut, Copenhagen, Denmark.

Antibodies against B. burgdorferi were detected using an IFA procedure similar to the one described for the Leptospira antibody analysis. Antigen slides were prepared using strain B. burgdorferi strain ACA1. Screening dilution was 1:50. The remaining process and secondary antibodies were as stated for the L. interrogans procedure. Antigen and positive controls from a dog (Canis familiaris) were kindly supplied by K. Bergstrøm, National Veterinary Institute (SVA) Sweden).

Antibodies against Seoul virus were detected using ELISA from Focus Diagnostics (Hantavirus IgG DxSelect™ Focus Diagnostics, California, USA, LOT-nr: 050072). The sera were diluted 1:100 and performed according to the manufactures instructions.

Follow Up on L. interrogans Results by Indirect Hemagglutination Assay

Additional serological analysis for antibodies against L. interrogans was performed using an indirect hemagglutination assay (IHA, Leptospirosis IHA Test Kit, Focus Diagnostics, California, USA, LOT-nr: 050620). The sera were diluted 1:50 and the analysis performed according to the manufactures instructions.

Due to results inconsistent with the L. interrogans antibody, an IFA test for possible inhibition of antibody-antigen binding was conducted. This included a dilution of the control provided by the manufacturer using test samples containing 80%, 50% and 20% control sample, where either buffer, sera from 3 MS patients positive and 3 negative IFA L. interrogans antibodies were added (sera no: 6, 8, 11, 16, 18 and 20). Sera samples were diluted to 1:50 and 10, or 40 μl added to the control antibodies to produce the required test volume of 50 μl. The microtiter plates were placed at 5° C. and re-evaluated after 24 hours.

### Results

Four samples were found to be positive for L. interrogans antibodies, cf. Table 5.

TABLE 5

| | | | | Antibodies against L. interrogans at sera dilution | | | | | |
|---|---|---|---|---|---|---|---|---|---|
| | | | | 1:40 | | 1:80 | | 1:160 | |
| Patient # | MS type | Sex | Age | Patoc | Icterhaem | Patoc | Icterhaem | Patoc | Icterhaem |
| 1 | P | M | 57 | 1 | N | N | N | N | N |
| 2 | P | F | 40 | 2 | 1 | B | N | N | N |
| 3 | P | F | 45 | B | N | N | N | N | N |
| 4 | P | M | 68 | B | 1 | N | B | N | N |
| 5 | P | M | 60 | 2 | B | B | N | N | N |
| 6 | PR | M | 54 | 4 | 4 | 4 | 3 | 2 | N |
| 7 | PR | M | 60 | B | B | N | N | N | N |
| 8 | PR | F | 39 | 4 | 4 | 3 | 3 | 1 | N |
| 9 | PR | M | 76 | 2 | 2 | N | B | N | N |
| 10 | PR | F | 53 | 1 | B | N | N | N | N |
| 11 | RR | M | 54 | 3 | 3 | 2 | 1 | B | N |
| 12 | RR | F | 35 | B | B | N | N | N | N |
| 13 | RR | F | 43 | B | B | N | N | N | N |
| 14 | RR | M | 45 | B | N | N | N | N | N |
| 15 | RR | F | 37 | B | N | N | N | N | N |
| 16 | RRE | m | 61 | N | N | N | N | N | N |
| 17 | RRE | f | 53 | 3 | 3 | 2 | 1 | B | N |
| 18 | RRE | m | 53 | N | N | N | N | N | N |
| 19 | RRE | m | 56 | B | N | N | N | N | N |
| 20 | RRE | f | 40 | N | B | N | N | N | N |

P: progressive,
PR: progressive with relapse,
RR: Relapsing remitting.
RRE: Relapsing Remitting in remission.

Three samples were undoubtedly negative. The result for both serovars Patoc and Ictehaemorhagia concurred, but serovar ictehemorhagia scored less than Patoc and more borderline positives were observed in serovar Patoc at 1:40 dilution than for serovar icterhaemorhagia. At dilution 1:160 two samples were positive, while no positive were found at dilution 1:320. Grouping the samples according to the presence of antibodies 1:40 dilution gives a clear differentiation between the four types of patients included in this study, cf. Table 6.

TABLE 6
Grouping of L. interrogans antibodies according to MS
type (Based on seradilution 1:40).

| | | Bordeline | |
|---|---|---|---|
| | | to weak | Strong positive |
| MS type | Negative | positive | response |
| Progressive | 0 | 5 | 0 |
| Progressive with relapse | 0 | 3 | 2 |
| Relapsing remitting in relapse | 0 | 4 | 1 |
| Relapsing remitting in remission | 3 | 1 | 1 |

Progressive MS patients all have low levels of antibodies, relapsing types of MS i.e. progressive MS with relapses or relapsing-remitting MS can have high levels of antibodies, while relapsing-remitting MS patients in remission constitute the only patient group free of even low levels of antibodies. There was no indication that age or sex had any influence on the presence of antibodies against L. interrogans.

All MS sera samples were 1+ in the IHA leptospira test, but negative controls also gave similar results. Thus all sera at dilution 1:50 were considered to be negative for antibodies in the IHA test. Manufacturer's control and the control provided from SSI (1:50) gave a score of 4+. Diluting the manufacturer's control antibodies with buffer reduced the score from 4+ to 3+ at 50% control and to 2+ at 20% control. The reduction in the score of the control was far greater when diluting with MS sera. The scores for all six dilutions with MS sera were 2+, 2+ and 1+ at 80%, 50% and 20% control, respectively. After 24 hours at 5° C. the score was higher for 20% control diluted with buffer than for 80% control diluted with sera from MS patients.

A single positive sample for B. burgdorferi sl was found (patient no. 1). No antibodies against Seoul virus was detected.

These results are reported in Table 7 below.

TABLE 7
results from IHA leptospirosis test including dilution series of control
with buffer and MS patients sera.

| | Control antibodies diluted with | | |
|---|---|---|---|
| | | Sera from IFA | Sera from IFA |
| | | L. interrogans | L. interrogans |
| | Buffer | antibody | antibody |
| | provided by | positive | positive |
| Dilutions | manufacturer | samples | samples |
| Pure control | 4+, (4+) | 1+ | 1+ |
| antibodies/sera (1:50) | | | |
| 80% control antibodies | 4+, (4+) | 2+, (1+) | 2+, (1+) |
| 50% control antibodies | 3+, (3+) | 2+, (1+) | 2+, (1+) |
| 20% control antibodies | 2+, (2+) | 1+, (Boarderline) | 1+, (1+) |

N = 20 for sera samples at 1:50 and n = 6 for control dilution series (three last rows).
Numbers in parenthesis is the results after 24 hours at 5° C.

### Discussion

The identification of four cases of leptisporosis appear to be higher than expected by chance alone. There are less than a hundred cases of leptospirosis recorded in the USA (Meites et al. 2004) and the chances that four of 20 samples should contain antibodies must be considered quite small. Prevalences of 15% have been reported from Baltimore (Vinetz et al. 1996), but as discussed later, this information may be biased. In any event, even a prevalence of 15% is not quite sufficient to allow for four cases in 20 samples.

In all analyses it was noted that the quality of the sera was extremely high. Thus, the B. burgdorferi sl analysis produced 19 clear and unmistakably negative results even at a dilution of 1:50. A similar observation was made in the Seoul virus analysis where just one of the samples had an absorbance higher than 1/10 of the cut off absorbance (data not shown).

In evaluating the score of antibodies it must also be considered that the true strain that may be responsible for generating antibodies is unknown. Therefore the level of cross-reactivity remains unknown. It is argued that serovar Patoc carries all the antigen of the genus Leptospira which is the reason that it is used for screening purposes (Lemcke et al, 2004). However, by definition, Patoc can only carry all known antigens and the responsible agent may belong to a subclass of L. interrogans not previous isolated. No information is available on any medical therapy that the patients may have been undergoing, and thus is cannot be excluded that the immune response is weaker in these sera than in a normal background. It can therefore not be excluded that antibodies observed at 1:40 are specific L. interrogans antibodies and not unspecific reactive antibodies.

The leptospirosis IHA test suggest that conventional leptospirosis can be distinguished from MS by combining the IHA and IFA tests for leptospiral antibodies. MS sera will be negative in IHA and positive in IFA, with the exception of Relapsing-Remitting MS patients in remission. It seems possible that there is a component in the MS sera that, irrespective of the presence of antibodies against L. interrogans, interferes with the hemagglutination process. The stronger binding in the IFA may be less sensitive. Still such interference suggests that also the score in the IFA may be reduced and that the true concentration of antibodies is higher than recorded by IFA. It could be speculated that the exact strain of L. interrogans has the highest level of interference with the component interfering with the IHA and hence it cannot be concluded that the highest score in the IFA is associated with the infecting organism.

The 1:40 dilution appears useful, when reviewing the observations for the four types of patients. The data generates an interpretable pattern where non-relapsing MS all have low antibody responses to L. interrogans. Relapsing MS can have higher levels of antibodies, while the only MS group without antibodies are relapsing remitting MS patients in remission. This may be interpreted as progressive MS being a chronic infection while relapsing remitting MS is not and that relapses are associated with high level of antibodies against L. interrogans.

The results are very clear in respect to Seoul Virus and B. burgdorferi sl. antibodies The single positive B. burgdorferi sample does not support a claim that B. burgdorferi sl is involved in MS. According to American Lyme Disease Foundation Inc. (www.adlf.com), Lyme borreliosis is known to be found in significant numbers on the west coast of USA and it seems likely that an MS patient should originate among the hundreds of Lyme borreliosis cases. Hence it is very clear that L. interrogans (or a bacterium strongly cross-reacting therewit) is the only bacterium, which can be accepted as a possible etiological agent of the agent included in this study.

Given the outcome of the serological study, a limited literature study was performed to determined whether the ecology of leptospirosis could be consistent with the epidemiological features of MS.

### Shared Ecological Features of Leptospirosis and MS

As mentioned above, Steiner was probably the first to propose that there was an association between a spirochete and MS (Blackman and Putnam, 1936). Steiner described both the spirochetes and granular structures, which was associated with the bacteria. He later described these as cyst from of the spirochete (Steiner, 1952). More importantly Hassain and Diamond (1939) stated that these spirochetes can be found in all MS patients, even though it was only demonstrated in 8 cases. Leptospira, like the rest of the spirochetes are not normally associated with cyst formation. Still, a compilation of known literature on the subject gives 200 articles published on the subject. One of these is a record of cyst formation in Leptospira (Inada et al. 1916). More recently Brorson et al., (2001) also pointed to a possible relation to a spirochetal organism. (A full discussion on the possibility of spirochetes being associated with MS is given by Marshall (1988)). Hence it seems that there are ample support for the idea that MS may be cause by a spirochete and that chronic infection may occur. Bacterial cysts may mediate the persistence of infection.

Prevalence of Leptospira in rats is found typically to be around 30% (10-70%) (Sunbul et al., 2001; Webster et al., 1995; Lilenbaum et al., 1993; Pereira and Andrade, 1988). Reports of prevalence of antibodies against Leptospira in humans are 25-72% in China (Pan and He, 1995), 25-35% in the slums of Rio de Janiero, Brazil (Pereira and Andrade, 1990) and 16% in Baltimore, USA (Vinetz et al. 1996), which suggests that Leptospira infections are common and common enough to function under the “Hygiene Hypothesis” (Strachan, 1989; Sheikh and Strachhan, 2004). The theory states that illness arises from a reduced childhood or infrequent exposure to the environmentally derived infections. Later, Gale (2002) suggested that the “Gatekeeper Hypothesis” i.e., that repeated early infections is needed to allow for regulatory T cells to control the immune response, could serve a possible mechanism. The results here could suggest that antibodies maintained through repeated L. interrogans infections will modify this response and reduce or prevent the manifestation of MS.

Leptospirosis pathology is primarily known for including renal failure (icteric), but also includes anicterict forms (Levett, 2001). Leptospirosis is argued to be underdiagnosed (WHO, 2003a; Lemcke et al. 2004). More over the great majority of (anicteric) leptospirosis are either sub clinical or very mild (Levett, 2001). Just as important, MS and leptospirosis share the clinical feature that incidence in children is low. In leptospirosis the age of 15 years seems to be considered as a threshold for the risk of clinical manifestations (WHO, 1999, Hannond et al., 2000; Planck and Dean, 2000) but cases among young children can be found (WHO, 1999). Thus there seems to be a shared feature relating to the threshold age of approximately 15 years of age.

Interpreting seasonality for MS in the current context is quite difficult because we have no hard information on the period of time from infection to first clinical manifestation in MS. Such comparisons are therefore essentially impossible. Still we may note that seasonality is a common feature of both leptospirosis and MS. In Denmark leptospirosis is most common in the autumn (K. Krogfelt Statens Serum Institut, Denmark, pers. comm.). 76% of first exacerbation in MS occurs in winter months while there is no seasonal variation in time of onset (Bisgard, 1990). Ocular involvement seems to be another shared characteristic of leptospirosis and MS. Eye lesions are common in the preliminary phase of MS, and share a seasonal periodicity similar to MS onsets (Ya-ping, 2000). Various forms of eye lesions are can be found in up to 85% of patient with confirmed leptospirosis (Martins et al., 1998) and optic neuritis, which is considered a key symptom for determining onset of MS occurs in 65% of leptospirosis infections (Mancel et al. 1999). Finally, equine recurrent uveitis, which currently is defined as an autoimmune disease due to antigen mimicry by Leptospira spp. (Lucchesi et al., 2002) underline that recurrence or relapses are a shared feature.

Long term periodicity in MS epidemiology has been reported from the Faeroe Island (Kurtzke and Heltberg, 2001). Also in more stable manifestation a periodicity of 5 years has been reported (Meyer-Reinecker and Buddenhagen, 1988; Jin et al. 2003), which in Sweden tends to be changing into longer irregular cycles (Jin et al. 2003). No detailed information exists on population cycles on R. norvegicus in Sweden or Denmark. A 10 year cycle has been proposed in England (Swift, 2001) and possibly a similar cycle exist in Denmark. It is impossible to know whether it has been shorter in previous centuries but it can be noted that microtine cycles in Sweden seem to fade out through the last decades (Hörnfeldt, 2004). More simply, the minimum frequency of exacerbation could be under immunological control in relapsing-remitting MS, where antibodies against Leptospira which may be maintained for 3-4 years (Levett, 2001) sets the minimum frequency by protecting against Leptospira attack. The duration from the disappearance of antibodies to the following infection is then determined by exposure to Leptospira, where periodic peaks in rat densities will tend to synchronize attack leading to cycles. It can thus not be excluded that long-term periodicity are similar in leptospirosis and MS.

Because Leptospira can be transmitted by semen in e.g., cattle (Heinemann et al., 2000) and rabbits (Kiktenko et al., 1976), this might be the case in humans as well. A possible sexual transmission may influence sex:ratio in MS and account for the differences between sex, which usually is 2 females for each male (Warren and Warren, 1993). It also mean that the prevalence of 16% antibodies against Leptopirosis in Baltimore USA, may be biased because these were drawn from a clinic for sexually transmitted diseases (Vinetz et al., 1996). Possibly the prevalence is lower in inner cities.

Import of cases of disease is within the normal range of considerations for leptospirosis and so are the imports of infectious agents. For Leptospira we may expect the fresh fruits and vegetables may harbour spirochetes, because Leptospira survives for up to 180 days in surface water in the temperature range of 7-36° C. (McDonough, 2001). R. norvegicus is suggested to be the only selective chronic carrier of the Leptospira ictehemorrhagia (Theirman, 1981). Alternative host for L. interrogans serovar ictehemorrhagia includes mice, raccoon, hedgehog, fox, woodchuck, skunks, muskrats and dogs), which may be a source of Leptospiral infections. Hence if MS is cause by this bacteria then (multiple) passage though alternative host may affect the virulence and lead to more benign cases. Such mechanism may be responsible for the variation in MS observed in e.g., Alberta, Canada (Warren and Warren, 1992)

### REFERENCES

Ascherio A, Munger K. L., Lenette E. T., Spiegelman D., Hernan M., Olek M. J., Hankinson S. E., Hunter D. J. 2001. Epstein-Barr virus and risk of multiple sclerosis: A prospective study. JAMA 286, 24: 3083-3088
Bisgard C. 1990. Seasonal variation in dissiminated sclerosis (Danish). Ugeskrift for laeger 152: 1160-1.
Blackman N. and Putnam T. J., 1936. Nature of the silver cells occurring in multiple sclerosis and other disease. Archives of Neurology and Psychiatry. 54-61.
Bloch, D. 1999. Vilini Súgdjor í Útnordri. Føroya Skúlabókagrunnur. Føroya Nátúrugrippasavn, Torshavn.
Brock T., Madigan. M., MArtinko J., Parker I., 1994. Biology of microorganism, 7ed. Prentice Hall International inc. pp. 765-766
Brorson, Ø., Brorson S., H., Henriksen T. H., Skogen, P. R. and Schøyen R., 2001. Association between multiple sclerosis and systic structures in the cerebrospinal fluid. Infection 29: 315-319.
Coleman P. G., Perry B. D., Woolhouse M. E. J., 2001. Endemic stability—a veterinary idea applied to human public health. Lancet 357: 1284-86
Conlon P., Oksenberg, J. R., Zhang J. and Steinman L. 1999. The immunobiology of multiple sclerosis: An Autoimmune disease of the central nervous system. Neurobiology of Diseases 6: 149-166.
Fritzsche, M 2002, Geographical and seasonal correlation of multiple sclerosis to sporadic schizophrenia. International Journal of Health Geographics http://www.ij-healthgeographics.com/content/pdf/1476-072X-1-5.pdf
Gale E. A. M, 2002. A missing link in the hygiene hypothesis. Diabetologia 45: 588-594.
Gratz N. (unknown date) Rodent reservoir and flea vectors of natural foci of plaque. WHO/CDS/EDC/99.2 Plaque manual: Epidemiology, Distribution, Surveillance and Control.
Hammond S. R., English D. R. and Mcleod J. G., 2000. The age-range of risk of developing multiple sclerosis. Brain 123: 968-974
Hassain G. B. and Diamond I B., 1939. Silver cells and spirochete-like formations in MS and other diseases of the central nervous system. Archives of Neurology and Psychiatry 41: 471-483
Hawkes C. H., 2002. Is multiple sclerosis a sexually transmitted infection. J. Neural Neurosurg Psychiatry 73: 439-443.
Heinemann M. B, Garcia J. F. Nunes C. M. Gregori F., Higa Z. M. Vasconcellos S. A., Richtzechain L. J. 2000. Detection and differentiation of leptospira spp serovars in bovine semen by polymerase chain reaction and restriction fragments polymorphism. Vet. Microbiol. 73, 4: 261-7.
Hörnfeldt B. 2004. Long term decline in numbers of cyclic voles in boreal Sweden: analysis and presentation of hypotheses. Oikos 107: 376-392.
Inada R. Ido. Y., Hoki R., Kanedo Ito., H., 1916. The etiology of infection and specific therapy of weils disease. Journal of experimental medicine. 23: 377-402
Jensen and Jespersen (Submitted). Five decades of tick-man interaction in Denmark—an analysis. Exp. Appl. Acarol.
Jin Y., Pedro-Cuesta J., Söderstrom M., Stawiarz L. and Link, H. 2000. Seasonal patterns in optic neuritis and multiple sclerosis: a meta analysis. Journal of the Neurological Sciences 181: 56-64.
Jongejan F., and Estrada-Pena A., 1999. Ticks Feeding on Humans: A Review of Records on Human-Biting Ixodoidea with Special Reference to Pathogen Transmission. Experimental and Applied Acarology 23, 9: 685-715.
Kaiser M., 1997. Fish farming and the precautionary principle: context and values in environmental science for policy. Foundations of Science 2: 307-341.
Kazmierski R., Wender M. Guzik P. and Zielonka D., 2004. Association of influenza incidence with multiple sclerosis onset. Folia Neurpathol. 42, 1: 19-23
Kiktenko V. S. Balashov N. G. Rodina V. N. 1976. Leptospirosis through insemination of animals. J Hyg Epidmiol Microbiol Immunol 21, 2: 207-13.
Kissler H., 2001. Is multiple sclerosis caused by a silent infection with malarial parasites? A historica-epidemiological approach: Part II. Medical Hypotheses 57: 292-301.
Koch-Henriksen N. Rasmussen S, Stenager E, Madsen M. The Danish Multiple Sclerosis Registry. History, data collection and validity. Dan Med. Bull. 2001 May; 48 (2):91-4.
Kurtzke J. and Heltberg, A. 2001. Multiple Sclerosis in the Faeroe Islands: An epitome. Journal of Clinical Epidemiology 54: 1-22
Lauer, K. 1991. The food pattern in geographical relation to the risk of multiple sclerosis in Mediterranean and Near East region. Journal of epidemiology and Community Health 45, 3: 251.
Leblebicioglu H., Sunbul M. Esen S. Eroglu C., 2003. Jarisch-Herxheimer reaction in Leptospirosis. European Journal of clinical microbiology and infectious diseases 22: 639.
Lemcke A., Rasmussen E., Glismann S., Krogfeldt K. A. (In press) Leptospirose i Danmark 1980-2002. Ugeskrift for Laeger. (Danish)
Levett P. N. 2001. Leptospirosis. Clinical Microbiology reviews 14, 2: 296-323.
Lilenbaum W., Riberio V., Martin E. and Bispo., 1993. Serological study for detecting anti-Leptospira antibodies in rattus norvegicus from Duque de Caixias, Rio de Janeiro, Brazil. Rev. Latinoam Microbiol 35 (4): 357-80. (Portuguese, abstract only)
Lucchesi P. M A, Parma A. E. Arroyo G. H., 2002. Serovar distribution of a DNA sequence involved in the antigenic relationship between Leptospira and equine cornea. BMC Microbiology. WWW.biomedcentral.com/1471-2180/2/3
Mancel E. Merien F., Pesenti L., Salino D., Angibaud G., Perolat P. 1999. Clinical aspects of ocular leptospirosis in New Caledonia (South Pacific) Australian and New Zealand Journal of opthalmology 27, 6: 380
Marshall V. 1988. MS is a chronic central nervous system infection by a spirochetal agent. Medical hypotheses. 25: 89-92
Martins M G, Matos K T, da Silva M V, de Abreu M T. 1998. Ocular manifestations in the acute phase of leptospirosis. Ocul Immunol Inflamm 6 (2): 75-9.
McDonough P. L., 2001. Leptospirosis in Dogs—current status. In Recent advances in Canine infectious diseases ed: Carmichael L. International Veterinary Informations Service (WWW.ivis.org), Ithaca, N.Y., USA.
Meites E., Jay M. T., Deesingsky S., Shieh W-j., Zaki S., Tompkins L. and Smith D. S., 2004. Remerging Leptospirosis, California. Emerging Infectious Diseases 10, 3 406-412.
Murphy F. A. (1998). Emerging Zoonoses. Emerging Infectious diseases 4, 3 (www.cdc.gov/ncidod/eid/vol4no3/murphy.htm)
Olsson, G. E., 2003 Ph.d. thesis: Nephropathia Epidemica and Puumala Virus Occurrence in Relation to Bank Vole (Clethrionomys glareolus) Dynamics and Environmental Factors in Northern Sweden, Department of Animal Ecology, Swedish University of Agricultural Sciences, SE-901 83, Umeå, Sweden
Pan Z. A. He Q. Y., 1995: The sero-epidemiological investigation of leptospirosis in Hainan Province. Zhonghua Liu Xing Bing Xue Za Zhi 16, 6:369-71. (Chinese, abstract only)
Pereira M. M and Andrade J. 1988. Epidemiological aspects of leptospirosis in a slum area in the city of Rio de Janeiro Brazil. Search for leptospirosis and specific antibodies in rodents. Trans R Soc Trop Med Hyg 82 (5):768-70
Pereira M. M and Andrade J. 1990. Human leptospirosis in a slum area in the city of Rio de Janiero, Brazil—a serological and epidemiological study. Mem inst Ostwaldo Cruz 85, 1: 47-52.
Picardeau M., Ren S., Saint Girons I., 2001. Killing effect and anti-toxin of the Leptospira interrogans Toxin-Antitoxin system in Escherichia coli. Journal of Bacteriology 183, 23: 6494-6497
Planck R. and Dean, D. 2000. Overview of the epidemiology, microbiology and pathogenesis of Leptospira Spp. In Humans. Microbes and Infection 2: 1265-1276.
Rohowsky-Kochan C. Dowling P. and Cook S. 1997. Antibodies to canine distemper virus in human sera. Journal of Neurological sciences 150, 1000, s118.
Rosati, G. 2001, The prevalence of multiple sclerosis in the world: an update. Neurol Sci. 22: 117-139.
Schmidt B., Arberer E., Stockenhuber C., Klade H., Breier F., and Luger A., 1995. Detection of Borrelia burgdorferi DNA by Polymerase Chain Reaction in the Urine and Breast Milk of Patients with Lyme Borreliosis. Diagnostics Microbiology and Infectious disease 21, 3: 121-128.
Sheikh A. and Strachan D. P. 2004. The hygiene theory: fact or fiction. Curr Opin Otolaryngol head neck surg 12, 3: 323-6.
Smith, R. L. & Smith T. M. 2002 Elements of Ecology, 5th edition, Benjamin Cummings
Sonenshine, D. E., 1991. Biology of ticks, vol. 1 and 2. Oxford University Press, New York.
Sparre P and Sibren C. V. 1998. Introduction to tropical Stock assessment. FAO fisheries technical Paper, DANIDA, Food and Agricultural organisation of the united states, FAO, Fiat Panis, ROME. (WWW.Fao.org/docrep/w5449e/w5449e00.htm), Chapter 9.
Sparre P and Sibren C. V. 1998. Introduction to tropical Stock assessment. FAO fisheries technical Paper, DANIDA, Food and Agricultural organisation of the united states, FAO, Fiat Panis, ROME. (WWW.Fao.org/docrep/w5449e/w5449e00.htm), Chapter 9.
Statens Skadedyrs laboratories 1965-1990. Årsberetning fra Statens Skadedyrs laboratories Statens skadedyrs laboratorie. (Danish)
Stanek G., O'connell S., Cimmino M., Aberer E., Kristoferitsch W., Granstrôm M., Guy E. and Gray J. 1996. Winer Klinische Wochenschrift 108, 23: 741-744.
Steiner G. 1952. Acute plaques in multiple sclerosis, their pathogenity, significance and the role of spirochetes as etiological factor. Journal of Neuropathy. 11: 343-72
Steriner G. 1952. Acute plaques in multiple sclerosis, their pathogenity, significance and the role of spirochetes as etiological factor. Journal of Neuropathy. 11: 343-72
Strachan D. P. 1989. Hay fever, hygiene and household size. BMJ 299:1259-60
Sunbul M Esen S., Leblebibioglu H., Hokelek M., Pekbay A., Eroglu C., 2001. Rattus norvegicus acting as reservoir of leptospira interrogans in the Middle Black Sea region, as evidenced by PCR and presence of serum antibodies to Leptospira strain. Scand. J Infect Dis 33 (12): 896-8
Swift J. D., 2001. Rat populations dynamics—is there a 10 year cycle. International Pest Control July/August: 157-159.
Tattevin P., Jauréguiberry S., and Michelet C., 2003. Meningitis as a possible feature of the Jarisch-Herxheimer Reaction in leptospirosis. European Journal of clinical microbiology and infectious diseases 22: 449
Theirman A. B. 1981. The Norway rat as a selective chronic carrier of Leptospira icterhemorrhagiae. J. Wildl. Dis. 17, 1: 39-43.
Vapalathi O., Mustonen J. Lundkvist A. Henttonen H., Plyusnin A. and Valheri A., 2003. Hantavirus infections in Europe. The Lancet Infectious Diseases 3, 10: 653-661.
Vinetz J. M., Glass G. E., Flexner C. E., Mueller P., Kaslow D. C., 1996. Sproradic Urban leptospirosis. Annals of Internal Medicine 125, 10: 794-798.
Warren S. A. Warren K. G. 1993. Prevalence, incidence and characteristics of multiple sclerosis in Westlock County, Alberta, Canada. Neurology 43: 1760-1763.
Webster J. P., Ellis W. A., Macdonald D. W., 1995. Prevalence of Leptospira spp. In wild brown rats (Rattus Norvegicus) on Uk Farms. Epidemiol infect. 114, 1: 195-201.
Wermuth L., Joensen P., Bunger N. and Jeune B. High prevalence of Parkinson's in the Faroe Islands. Neurology 49, 2: 426-432
WHO 1999. Weekly Epidemiological Record (leptospirosis worldwide) 74: 237-244
WHO 2003a. Human leptospirosis: Guidance for diagnosis, surveillance and control. Available on: http://www.who.int/csr/don/en/WHO_CDS_CSR_EPH_{—}2002.23.pdf
WOO T H et al., “Rapid distinction between Leptospira interrogans and Leptospira biflexa by PCR amplification of 23S ribosomal DNA”, FEMS Microbiol Lett. 1997 May 1; 150 (1):9-18.
Woolhouse M. E. J. 2002, Population biology of emerging and re-emerging pathogens. Trends in microbiology 10, 10: 3-7.
Woolhouse M. E. J., 2001. Populations biology of emerging and re-emerging pathogens. Trends in Microbiology, 10, 10:3-7

1. A method for treating or ameliorating multiple sclerosis (MS) in a human subject suffering from MS, the method comprising active immunization of said subject with an immunogenic agent that induces a therapeutically effective immune response against antigenic determinants derived from Leptospira, said immunogenic agent comprising a specific immunogen.

2. A method for preventing multiple sclerosis (MS) in a human subject, the method comprising active immunization of said subject with an immunogenic agent that induces a protective immune response against Leptospira, said immunogenic agent comprising a specific immunogen.

3. The method according to claim 1, wherein the Leptospira is L. interrogans.

4. The method according to claim 1, wherein the specific immunogen is selected from the group consisting of
a) a preparation of a live Leptospira species which is non-pathogenic in humans and which preferably cross-reacts immunologically with L. interrogans,
b) a preparation of killed or inactivated L. interrogans or killed or inactivated bacteria from a cross-reactive Leptospira species or strain,
c) an antigen fraction isolated from L. interrogans or from a cross-reactive Leptospira species or strain,
d) a preparation of at least one antigen comprising immunodominant epitopes derived from L. interrogans or from a cross-reactive Leptospira species or strain,
e) a preparation comprising at least one anti-idiotypic antibody reactive with the idiotype of an antibody that binds to an immunodominant epitope derived from L. interrogans or from a cross-reactive Leptospira species or strain,
f) a preparation comprising at least one mimotope of an immunodominant epitope derived from L. interrogans or from a cross-reactive Leptospira species or strain, and
g) a preparation of nucleic acids encoding and being capable of effecting in vivo expression from the subject's cells of at least one immunodominant protein antigen derived from L. interrogans or from a cross-reactive Leptospira species or strain.

5. The method according to claim 4, wherein the specific immunogen is selected from the group consisting of d, e, and f, and is coupled to
at least one first moiety which effects targeting of the modified molecule to an antigen presenting cell (APC) or a B-lymphocyte, or
at least one second moiety which stimulates the immune system, or
at least one third moiety which optimizes presentation of the active principle to the immune system.

6. The method according to claim 5, wherein the first moiety is a substantially specific binding partner for a B-lymphocyte specific surface antigen or for an APC specific surface antigen, such as a hapten or a carbohydrate for which there is a receptor on the B-lymphocyte or the APC.

7. The method according to claim 5 wherein the second moiety is selected from the group consisting of a cytokine, a hormone, and a heat-shock protein.

8. The method according to claim 5, wherein the cytokine is selected from, or is an effective part of, interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), and the heat-shock protein is selected from, or is an effective part of any of, heat-shock protein 70 (HSP70), heat-shock protein (HSP90), heat-shock cognate 70 (HSC70), glucose related protein (GRP94), and calreticulin (CRT).

9. The method according to claim 5, wherein the third moiety is of lipid nature, such as a palmitoyl group, a myristyl group, a farnesyl group, a geranyl-geranyl group, a GPI-anchor, and an N-acyl diglyceride group.

10. The method according to claim 5, wherein the specific immunogen includes at least one heterologous MHC-Class II binding peptide sequence capable of stimulating T-helper lymphocytes.

11. The method according to claim 10, wherein the MHC-Class II binding peptide sequence is comprised in an immunogenic carrier protein or is present in the form of a universal T-helper lymphocyte epitope (T_{H} epitope).

12. The method according to claim 11, wherein the universal T-helper epitope is selected from the group consisting of a natural universal T_{H} epitope and an artificial MHC-II binding peptide sequence.

13. The method according to claim 12, wherein the natural T_{H} epitope is selected from the group consisting of a Tetanus toxoid epitope, a diphtheria toxoid epitope, an influenza virus hemagluttinin epitope, and a P. falciparum CS epitope.

14. The method according to claim 5, wherein at least two copies of the specific immunogen is covalently or non-covalently linked to a carrier molecule capable of effecting presentation of multiple copies of antigenic determinants from the specific immunogen.

15. The method according to claim 1, wherein the immunogenic agent further comprises an immunological adjuvant.

16. The method according to claim 15, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; calcium adjuvants such as calcium phosphate; DNA adjuvants; γ-inulin; and an encapsulating adjuvant.

17. The method according to claim 1, wherein an effective amount of the immunogenic agent is administered to the subject via a route selected from the group consisting of the parenteral route such as the intracutaneous, the subcutaneous, and the intramuscular routes; the peritoneal route; the oral route; the buccal route; the sublinqual route; the epidural route; the spinal route; the anal route; and the intracranial route.

18. The method according to claim 17, wherein the effective amount comprises between 0.5 μg and 5,000 μg of the specific immunogen.

19. The method according to claim 4, wherein the specific immunogen is g and the nucleic acids are introduced into the animal's cells, thereby obtaining in vivo expression by the cells of the nucleic acid(s) introduced.

20. The method according to claim 19, wherein the nucleic acids introduced is/are selected from the group consisting of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in chitin or chitosan, and DNA formulated with an adjuvant such as the as: an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; calcium adjuvants such as calcium phosphate; DNA adjuvants; γ-inulin; and an encapsulating adjuvant.

21. The method according to claim 19, wherein the nucleic acids are administered intraarterially, intraveneously, or via a route selected from the group consisting of the parenteral route such as the intracutaneous, the subcutaneous, and the intramuscular routes; the peritoneal route; the oral route; the buccal route; the sublingual route; the epidural route; the spinal route; the anal route; and the intracranial route.

22. The method according to claim 1, wherein the immunogenic agent comprises a pharmaceutically acceptable carrier, vehicle or diluent.

23. The method according to claim 1, wherein the immunization includes a primary immunization followed by at least one booster immunization.

24. The method according to claim 23, wherein the immunogenic agent used in the primary immunization and that used in the at least one booster immunization are identical.

25. The method according to claim 23, wherein the immunogenic agent used in the primary immunization and that used in the at least one booster immunization are non-identical.

26. A method for treating or ameliorating multiple sclerosis (MS) in a human subject suffering from MS, the method comprising administering a therapeutically effective amount of an antibiotic exhibiting bacteriotoxic or bacteriostatic effect on L. interrogans.

27. The method according to claim 26, wherein the antibiotic is capable of interfering with signalling between bacteria.

28. The method according to claim 26, wherein the antibiotic is selected from the group consisting of antibodies or fragments thereof, bacitracin, cephalosporins, cycloserine, penicillins, ristocetin, vancomycin, amphotericin B, colistin, imidazoles, nystatin, polymyxins, chloramphenicol, erythromycins, lincomycins, tetracyclines, aminoglycosides, nalidixic acid, novobiocin, pyrimethamine, rifampin, sulfonamides, and trimetoprim.

29. The method according to claim 28, wherein the antibiotic is capable of entering the CNS from the vascular system.

30. The method according to claim 29, wherein the antibiotic is a tetracycline.

31. The method according to claim 26, wherein the antibiotic is administered in combination with a further treatment regimen, which reduces or modulates the pathogenesis, said further treatment regimen being selected from the group consisting of an anti-inflammatory treatment regimen, treatment with a Leptospira toxin binding compound, treatment with a compound that inhibits Leptospira toxin production, and treatment with a compound that directly or indirectly promotes the degradation of Leptospira toxin.

32. The method according to claim 26, wherein the antibiotic is administered during or shortly after an MS attack.

33. A method for determining whether a person is suffering from MS or has an increased risk of attracting MS, the method comprising subjecting a sample obtained from the person to a test that determines whether or not the sample contains material derived from L. interrogans, a positive determination indicating that the person has a significantly increased risk of MS compared to a subject without a positive determination.

34. The method according to claim 33, wherein the test is selected from the group consisting of an immunoassay and an assay that utilises a molecular amplification procedure such as PCR.

35. A method for assessing the risk that a person is suffering from MS or will attract MS, the method comprising subjecting a sample obtained from the person to a test that determines the presence, in the sample, of antibodies generally reactive with Leptospira and antibodies specifically reactive with L. interrogans.

36. The method according to claim 35, wherein the risk is assessed as increased if the test reveals that the sample comprises antibodies that are generally reactive with Leptospira and antibodies that are specifically reactive with L. interrogans.

37. The method according to claim 35, wherein the risk is assessed as decreased if the test reveals that the sample comprises antibodies that are generally reactive with Leptospira but no antibodies that are specifically reactive with L. interrogans.

38. The method according to claim 35, wherein the risk is assessed as average if the sample comprises antibodies that are generally reactive with Leptospira and antibodies that are specifically reactive with L. interrogans.

39. A method for assessing the risk that a person is suffering from MS or will attract MS, the method comprising subjecting a sample obtained from the person to a test that establishes whether the person's alternative complement pathway is capable of lysing Leptospira.

40. (canceled)

41. The method according to claim 33, wherein an indirect hemagglutination assay (IHA) for determination of Leptospira is used as a further control.

42. The method according to claim 41, wherein the presence of a Leptospira positive finding in any other test than IHA and a Leptospira negative finding in the IHA is an indication that the patient suffers from MS or has an increased risk of attracting MS.

43. The method according to claim 33, wherein, if the sample is found to be derived from an MS patient, the type of MS is determined on the basis of the test results.

44. A method for monitoring the progress of MS in a patient, the method comprising subjecting a sample obtained from the patient to a test that quantitatively determines L. interrogans material in the sample and comparing the determination with determinations performed on later samples from the same patient.

45. A method for monitoring the progress of MS in a patient, the method comprising a quantitative determination of antibodies specifically reactive with L. interrogans in a sample obtained from the patient and comparing the determination with equivalent determinations performed on later samples from the same patient.

46. (canceled)

47. (canceled)

48. A pharmaceutical package comprising at least one container comprising an immunogenic agent capable of inducing protective immunity in humans against L. interrogans and instructions for using the immunogenic agent for treatment or prophylaxis of humans against MS.

49. A pharmaceutical package comprising at least one container comprising an antibiotic capable of exerting a bacteriotoxic or bacteriostatic effect on L. interrogans and instructions for using the antibiotic for treatment or prophylaxis of humans against MS.

50. A pharmaceutical kit, comprising at least one container comprising an immunogenic agent capable of inducing protective immunity in humans against L. interrogans and at least one container comprising diagnostic means that can react with L. interrogans material or react with antibodies reactive with L. interrogans.

51. The method according to claim 2, wherein the Leptospira is L. interrogans.

52. The method according to claim 2, wherein the specific immunogen is selected from the group consisting of
a) a preparation of a live Leptospira species which is non-pathogenic in humans and which preferably cross-reacts immunologically with L. interrogans,
b) a preparation of killed or inactivated L. interrogans or killed or inactivated bacteria from a cross-reactive Leptospira species or strain,
c) an antigen fraction isolated from L. interrogans or from a cross-reactive Leptospira species or strain,
d) a preparation of at least one antigen comprising immunodominant epitopes derived from L. interrogans or from a cross-reactive Leptospira species or strain,
e) a preparation comprising at least one anti-idiotypic antibody reactive with the idiotype of an antibody that binds to an immunodominant epitope derived from L. interrogans or from a cross-reactive Leptospira species or strain,
f) a preparation comprising at least one mimotope of an immunodominant epitope derived from L. interrogans or from a cross-reactive Leptospira species or strain, and
g) a preparation of nucleic acids encoding and being capable of effecting in vivo expression from the subject's cells of at least one immunodominant protein antigen derived from L. interrogans or from a cross-reactive Leptospira species or strain.

53. The method according to claim 52, wherein the specific immunogen is selected from the group consisting of d, e, and f, and is coupled to
at least one first moiety which effects targeting of the modified molecule to an antigen presenting cell (APC) or a B-lymphocyte, or
at least one second moiety which stimulates the immune system, or
at least one third moiety which optimizes presentation of the active principle to the immune system.

54. The method according to claim 53, wherein the first moiety is a substantially specific binding partner for a B-lymphocyte specific surface antigen or for an APC specific surface antigen, such as a hapten or a carbohydrate for which there is a receptor on the B-lymphocyte or the APC.

55. The method according to claim 53 wherein the second moiety is selected from the group consisting of a cytokine, a hormone, and a heat-shock protein.

56. The method according to claim 53, wherein the cytokine is selected from, or is an effective part of, interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), and the heat-shock protein is selected from, or is an effective part of any of, heat-shock protein 70 (HSP70), heat-shock protein (HSP90), heat-shock cognate 70 (HSC70), glucose related protein (GRP94), and calreticulin (CRT).

57. The method according to claim 53, wherein the third moiety is of lipid nature, such as a palmitoyl group, a myristyl group, a farnesyl group, a geranyl-geranyl group, a GPI-anchor, and an N-acyl diglyceride group.

58. The method according to claim 53, wherein the specific immunogen includes at least one heterologous MHC-Class II binding peptide sequence capable of stimulating T-helper lymphocytes.

59. The method according to claim 58, wherein the MHC-Class II binding peptide sequence is comprised in an immunogenic carrier protein or is present in the form of a universal T-helper lymphocyte epitope (T_{H} epitope).

60. The method according to claim 59, wherein the universal T-helper epitope is selected from the group consisting of a natural universal T_{H} epitope and an artificial MHC-II binding peptide sequence.

61. The method according to claim 60, wherein the natural T_{H} epitope is selected from the group consisting of a Tetanus toxoid epitope, a diphtheria toxoid epitope, an influenza virus hemagluttinin epitope, and a P. falciparum CS epitope.

62. The method according to claim 53, wherein at least two copies of the specific immunogen is covalently or non-covalently linked to a carrier molecule capable of effecting presentation of multiple copies of antigenic determinants from the specific immunogen.

63. The method according to claim 2, wherein the immunogenic agent further comprises an immunological adjuvant.

64. The method according to claim 63, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; calcium adjuvants such as calcium phosphate; DNA adjuvants; γ-inulin; and an encapsulating adjuvant.

65. The method according to claim 2, wherein an effective amount of the immunogenic agent is administered to the subject via a route selected from the group consisting of the parenteral route such as the intracutaneous, the subcutaneous, and the intramuscular routes; the peritoneal route; the oral route; the buccal route; the sublinqual route; the epidural route; the spinal route; the anal route; and the intracranial route.

66. The method according to claim 65, wherein the effective amount comprises between 0.5 μg and 5,000 μg of the specific immunogen.

67. The method according to claim 52, wherein the specific immunogen is g and the nucleic acids are introduced into the animal's cells, thereby obtaining in vivo expression by the cells of the nucleic acid(s) introduced.

68. The method according to claim 67, wherein the nucleic acids introduced is/are selected from the group consisting of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in chitin or chitosan, and DNA formulated with an adjuvant such as: an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; calcium adjuvants such as calcium phosphate; DNA adjuvants; γ-inulin; and an encapsulating adjuvant.

69. The method according to claim 67, wherein the nucleic acids are administered intraarterially, intraveneously, or via a route selected from the group consisting of the parenteral route such as the intracutaneous, the subcutaneous, and the intramuscular routes; the peritoneal route; the oral route; the buccal route; the sublinqual route; the epidural route; the spinal route; the anal route; and the intracranial route.

70. The method according to claim 2, wherein the immunogenic agent comprises a pharmaceutically acceptable carrier, vehicle or diluent.

71. The method according to claim 2, wherein the immunization includes a primary immunization followed by at least one booster immunization.

72. The method according to claim 71, wherein the immunogenic agent used in the primary immunization and that used in the at least one booster immunization are identical.

73. The method according to claim 71, wherein the immunogenic agent used in the primary immunization and that used in the at least one booster immunization are non-identical.

74. The method according to claim 35, wherein an indirect hemagglutination assay (IHA) for determination of Leptospira is used as a further control.

75. The method according to claim 39, wherein an indirect hemagglutination assay (IHA) for determination of Leptospira is used as a further control.

76. The method according to claim 74, wherein the presence of a Leptospira positive finding in any other test than IHA and a Leptospira negative finding in the IHA is an indication that the patient suffers from MS or has an increased risk of attracting MS.

77. The method according to claim 75, wherein the presence of a Leptospira positive finding in any other test than IHA and a Leptospira negative finding in the IHA is an indication that the patient suffers from MS or has an increased risk of attracting MS.

78. The method according to claim 35, wherein, if the sample is found to be derived from an MS patient, the type of MS is determined on the basis of the test results.

79. The method according to claim 39, wherein, if the sample is found to be derived from an MS patient, the type of MS is determined on the basis of the test results.
